SEQUENCE LISTING (47 sequences)

SEQ ID NO: 1 - protein, 218 aa, Zea mays
MAEETDTRPASAGSRGRPAPEDDDREEGEITDLACAPSPPATHPLEHSWTFWFDNPQSKSKQAAWGSSIRPIHTFSTVEEFWGLYNNINHPSKLIVGADFHCFKNKIEPKWEDPICANGGKWTISCGRGKSDTFWLHTLLAMIGEQFDYGDEICGAVVSVRGKQERIAIWTKNAANEAAQVSIGKQWKELLDYKDSIGFIVHDDAKKMDKGLKNRYTV

SEQ ID NO: 2 - protein, 220 aa, Zea mays
MADEIDTRPASAGSRGRPAHATEDDDREEGEIAADAPSPALPAAHPLEHSWTFWFDNPHGKNKMAAWGSSIRPIHTFSTVEDFWGLYNNIHHPSKLIGGADFHCFKNKIEPKWEDPICANGGKWTISCGRGKSDTLWLHTLLAMIGEQFDYGDEICGAVVSVRSKQERIAIWTKNAANEAAQISIGKQWKEFLDYKDSIGFIVHDDAKKMDKGLKNRYTV

SEQ ID NO: 3 - protein, 216 aa, Zea mays
MAEVEAPATAVEAPAAAVATTTPEVAAPEAGAATEAKGPHKLHRQWTFWYDIQTKTKSGAAWGTSLKKAYTFDTVEEFWSMYDQIFRPSKLSGNADFHLFKAGVEPKWEDPECANGGKWTVPCNRKATFETMWLETLMALIGEQFDETEDICGIVASVRARGDKLALWTRTASNEAVQVNIGKKWKDVIDYNDKITYTFHDDSKRDKPSRGGRYTV

SEQ ID NO: 4 - protein, 210 aa, Zea mays
MAEVEAPAAAVAAATPEVATVTEGGAATEAKGPHKLHRQWTFWYDIQSKPKPGAAWGTSLKKAYTFDTVEEFWGLYDHVFRPSKLPGTADFHLFKAGVEPKWEDPECANGGKWTVPCNRKPTFETMWLETLMALVGEQFDETDDICGIVASVRPRGDKLALWTRTASNEAVQVNIGKKWKDVIDYNDKIIYTFHDDSRRDKPSRGGRYTV

SEQ ID NO: 5 - protein, 218 aa, synthetic construct
  REGION 1..218: note = eIF4E1 variant
MAEETDTRPASAGSRGRPAPEDDDREEGEITDLACAPSPPATHPLEHSWTFWFDNPQSKSKQDDWGSSIRPIHTFSTVEEFWGLYNNINHPSKLIDRAGFHCFKNKIEPKWEDPICANGGKWTISCGRGKSDTFWLHTLLAMIGEQFDYGDEICGAVVSVRGKQERIAIWTKNAANEAAQVSIGKQWKELLDYKDSIGFIVHDDAKKMDKLLKNRYTV

SEQ ID NO: 6 - protein, 220 aa, synthetic construct
  REGION 1..220: note = eIF4E2 variant
MADEIDTRPASAGSRGRPAHATEDDDREEGEIAADAPSPALPAAHPLEHSWTFWFDNPHGKNKMDDWGSSIRPIHTFSTVEDFWGLYNNIHHPSKLIDRAGFHCFKNKIEPKWEDPICANGGKWTISCGRGKSDTLWLHTLLAMIGEQFDYGDEICGAVVSVRSKQERIAIWTKNAANEAAQISIGKQWKEFLDYKDSIGFIVHDDAKKMDKLLKNRYTV

SEQ ID NO: 7 - protein, 216 aa, synthetic construct
  REGION 1..216: note = eIFiso4E1 variant
MAEVEAPATAVEAPAAAVATTTPEVAAPEAGAATEAKGPHKLHRQWTFWYDIQTKTKSGDDWGTSLKKAYTFDTVEEFWSMYDQIFRPSKLSGRADFHLFKAGVEPKLEDPECANGGKWTVPCNRKATFETMWLETLMALIGEQFDETEDICGIVAKVRAEGDKLALWTRTASNEAVQVNIGKKWKDVIDYNDKITYTFHDDSKRDKPSRGGRYTV

SEQ ID NO: 8 - protein, 210 aa, synthetic construct
  REGION 1..210: note = eIFiso4E2 variant
MAEVEAPAAAVAAATPEVATVTEGGAATEAKGPHKLHRQWTFWYDIQSKPKPGDDWGTSLKKAYTFDTVEEFWGLYDHVFRPSKLPGRADFHLFKAGVEPKLEDPECANGGKWTVPCNRKPTFETMWLETLMALVGEQFDETDDICGIVAKVRPEGDKLALWTRTASNEAVQVNIGKKWKDVIDYNDKIIYTFHDDSRRDKPSRGGRYTV

SEQ ID NO: 9 - DNA, 10520 bp, Zea mays
  source 1..10520: mol_type = genomic DNA; note = eif4e1 genomic sequence with 3' and 5' UTR
atgtttggtttctttagtctaggggctaaagtttagttaggggactaaagtttagtctctaacatgtttggttttaggggctaaaaatagtaagaatataataaatgactcataaaagattaaaatggcctttaacattctcctgctattagtacaattgaactaaatgaggggtaaatgtggaattaatatggtttagtcccttttagcacatatgtgaaggactaaagactaaatcattttagtccatattttagtcctagtgtttgacaaaaaaaagactaaatagaactaaaaactagagactaatctttagttcctctaaccaaacaccacctaatccatccacatgaaagacgatgacacaatacatatctctttaactgccaccaatgccataatacgtgctactcgtgtaggacatgcacacgttggactaggcatgagcaggagcggagcgctcggtgcgcaagccataccttgatttggcagaccaaaagacctttggtcttggcctcgcgtccgcctgctactccttttctccgctgctcgcctaatcactagcttctccaccgttggttgtcgctcgttcgctctacgacgcgcgaggcatggccacacatgtgcctagtcgtgcggccatgccctccatgcttcccctcgatagcgtcgagtgcgttgcggctgttcatactacttcaggcgtctccctagtgtcgagcacttgctagttgactacgtaatgaggtcttggtcaataacccgtgtgattggttgccgcatgttagtagcctggttcggcttggccagcacatgaggagccatgctcatatggccaggctagcttcatacagtagactattgtttggttgtttgcattagtgtattcaggttagatataactcttgtttggttggctgtatcagaacaaagatctgataaaataaataaataaataaataaatctttaaaatttatatgataagtgcactctaattttgttcataaaaatatcaagaacgtagatctgaagttagcttagttattgaacatttcaactaagatatatgataaaaaaacaaaataaaaaaaatatttaggaacacgtatcgaggctctcatatgcatgcatgcagcctgcatccgcagcgacgctcctcagccgtgtacgatgggagcctggctaggaagcaaatcttgcatgtgtggatcctggttgatgcatgcaaggaaccaatcaagttatgctttgaggatccgcggctacaggtgatcggtacatgcaaggaaccaaacatacggaacatgaatgtccttatgggtacgcattccagatctggtttcccattggatgcttagctcttgagatgcttgtctctgtattatatacatgtataaaacttgtgacatctcaaattttgtgtgtccttcaccactgggcatgactataaccacaacactcgaacctagcgctacaaacatagggcccctgctatatcacataccctctccttgctgacgatggcatattgttgaatcagatcatctaaaaggccagagttattacgctcttatagaagttatggttgatagttgtttcaactaaataacttttagcctttttaagccgtgagagattcctgaaaatatgattctatctttgagccaaggtttaaaatagcgggctacagcggcgctatagcctggctatagcgttttgggagaccaaaagctacgatataccattttcacgcgctatatcagctatgagcgctatagctgaaaatagcgccgctaaatacaatagcgctagcgccaaatagctgatgctatttctgttttctagagtgtagggatgaaagtggtaatccgaattgttaggacaaatttaatattttaaagtagatatgtataaaatttgatgttgatcttttcttgtgttatcaagcacattagtacaaatatgaataaaatattacataaaatgttttatgtatgatttggtccctacaacacaaatagttgaaaaaattaccaaatttgtttccgaatccataccgaagtttatatctattatttaagaaaatataggatgaatttaaggcttatcttttatgaatttttactagttggatgttaaaataaagagtacaaatttgtattctatattctatatcctatttattcgcaatcaaagaaaaacgactaaaaaactgattaccgaataaataccgtttccgaccgttttcatccctactagagtgggccaaaaatagagcaatacattgaattcaatagtccatgaaggcccaaaactgaacctaaagtcatctgctgagagggacaaccggacaagcactaagccacatccctgaaacctcaaagaaacggcggcagcggtggcgcgcctgcatgctggtgctgggcacggctacatagggctccagttcgtgaccgcgaggcgaggagaggcaacctgacggaggtgttcaggtgctggcgtggtccttcgtccttgcttagttccttctcttgcttcctagcttcttgctgatagctagctgtccacactattgatctaatgcttgaggacatgggaaagattaagcaagtagatgaaacaattgtgagggcaagatctttgactacattcctttattcccatacaagggttttagcactcatgaggaaattccttggtaaggatctagtgcgtgctggtattacacgcttttctacaacctatttgaatttgaaaagcatgttaggcaacaagaaagagttaggaaagctgtttagatcagatgaactcaatgaaatgggttacttgaaaaaggataagggaaagaacgctagcaaaatagtgaggtctgatacattttgggaaaatgttgattgtgctgtcaatttttttgaaccactagctaatgtacttagaaggatgaatagtgatgtaccagccatgggtttcttatatggttgtttgttggatgcaaagaatgatattgccaaaagatttaacaatgttgaacgttgtttcaaaactatttgggaaattattgataagaggtgggataataagttgaagactcctttacatttggctggttattacttgaatctgtactactattatccaaataaattagagattgagttggatggaacatttagagagggtctagtatcctgtatcacaaagatggttcaagaggttgaaatccaagacaagataatggatgagctcaacatgtaccaggatgggatgggatcgtttggaaaagatattgccacaaggtagaggaggaatgaaaattttaatccaagtgattgaaaatctgacttctcttttcattagcctaatttcacatgtattcattaggccaatctaaatactgatactctactttttatgcagctaaatggtggttgaaccatggaaatagttctccaaatcttagaaagctagcaataaaaattttaggcttaacatgtagctcatcagcatgtgaaaggaattggagtgattttgaaaaggtaagaacagattgtgcaattttcacaattgcttgttactagagcagtagagttgtctcttatgtacttatctaaaatggtcatggtttctataaatctttataggttgactcaaagataaggagcatacttctccatgataggatgagggaccttgtgttcgtcaaatttaactcaaagttgaagcaaaaaaaagagaacaaatcaagagacccaattgagaagcaggttcctgatatcctagaagatagtggcaatgagtggatcactagagttgcatcaattgaacaagaacaagcacaaaacactgaatttgttgaagctacaaatgcacaaggtcagtcatcaaggacaacaatagaaacgggaaaaaggaagagagaaattcagcctagaagaaagacaaggacaaggaagctcattgatattaatgaatgcaatgatgatgaacctaagcttgcttcatcctctgattcagaagaagatgatgcacatgttgacatggatcaagagtcctcaagttctcattcttctgattcggataactccgtgtttgatgaatagaatgcactagtagtagttcacttacactattgcactaataataattttgttatgtaatggatgtctggatcatggtattatgtagctaacttgctacaatgctaccttggtgaactgatgaactacttgtttgctattgtagtaagtagtattttaaaagttggaactattgaattgtttgctgctaaactcttttggcctctgtgttgtttgtttaaggctttaccgctttattaagctgctttccagtctacagctgctcgctgtttggtgtttgctaaggtgtcacaatgtcaactattgattgaatgctgcattgctgcatatgctacaattgttggatggttgtacaactccaactttgactgtgttattgtcagtcttattgactatgtctgtgttattttttacaaatccactatttgctatagctacgctatacctgcgctatagcctcgtagcctaaaaaaatgttgccgctagtttgcatagcccgctattttaaaccttgctttgagctatgagaaaactgccgcgagctggtcgttttggaaaagctaaaatataattctataagagcatgatagcttgtaatacgaataatagaaatatggaaagtctaaggtaaggggtgattctaaattagactagaggaatttatagatttttaagaatctattctgctgttgtacctattttgttgagattccagttttttgacagtgaatctaatcctttgaatattttttttcataaatctacaattcggacagtgttataatgaatttgacgagaagctggtctatcccaaccacctaccatgggcctggcctggcctggcccatttctatgagaagagaggaggagaactgaagggatcaaagggaaaggaaacctagacaagcgaggcggcggccccgcgttctccgtctccgtctccgtctccgtccgcccggcgtaaagaaaagcctctctcaccacagccgccacgccatggccgaggagaccgacaccaggcctgcctccgcgggctcccgaggccgccccgcgcccgaggacgacgacagggaggagggcgagatcaccgacctcgcctgcgctccttccccgcccgccacgcacccgctcgagcactcctggaccttctggttcgacaacccgcagagcaagtccaagcaggccgcctgggggagctccatccgccccatacacaccttctccaccgtcgaggaattctgggggtgcgttcattctcttcagattcgccctccctcgtccttctatcctcttcccctgatcgcatcccactgtttctcttttccgttacaaatcctccgaccgagggccgccgcatgctcatgtaattggtaaattaattcggtgactagtgctagccacttagatgttacgtaaatctatatatgagcccaacctgtcacccttcagttcacggatccgagccgaaactcttgacatatcctagctgctactgttgccttttgattgagtactccttccgtccaaaatgtaaggggttttggctctctttttcccgtctacatccatatccatgttaatgaatctagacacatattcaaaccacatacatgatatataatatttggatcagtaaaaggttagaactccttacaatttgggaccgtggagtaaataagagtaaagttcacttctgatccttaaacttttctgctgtgtcaattctgtacatcaaattttaaagtatgtctcttaagagctcaatctctgtttgagtctctattaagtcctaacaggttgctgtggcccacatgcagtcgttgaggaagaagagagaagggtggatggggtgggggtctaacagaccacaacgactgacatataagccacgacagcccgttaggatgtaatcgaacagagattaagcccttaaaagacatactttaaaagtttatggatcagattgacacagccagacagatttaaggaccggaagtgcactttactcgagtaaataataaattgagcctccaacgtcacacaaaaccttttattctacatagatttgtgaactctttggaggtttcatttagggcatgtaccctgcatattaatgcaccatgccttaagatgtgtcaaagagcttcttcaataaggtgtctcgaaatagtgtcctaaaaattaaaacactacattatttaaagtgtttagaacactaaaaacaaattgaggtccaatagttaagtcctaaatcatgtattttaggaagtaaattacattttttttaataaaaaaatgctaagaataatgtagaaaacaaggatagatcactaacatggggtgtagtacatttgggtcacttcatggtatgtcctatatttgtttgataaaaatttgtaaaatagagtactgttggagccgttttttattatttaagccctatattttaagcccatttatgtgggttatactagtgaagttgtttcgttgattggaaacctgctattgttcacaaccaatatcagttttaccccttctctcctgataacttctctccttctgataagtggtcttttttctagtttcttgatggagcagtggtttgagacatctgctgatctctgcttcattccttttacatcctcgtacagctttaacttccatttattaatgatattccaattccaactaatggatctgtgctttactaggctttacaataacattaatcaccctagcaaattgatcgtgggagctgatttccattgcttcaagaacaagatcgagccaaaatgggaagaccctatttgtgcgaatggtggtaaatggaccatcagctgtggaagaggaaaatctgatacattttggctgcacactgtaagcaattctattttatcagaaagataactttgttttgcacatctctccttaacacgtcctccaccttatattctttcaactgtcctttgtttagctgctggctatgattggtgaacaattcgactatggtgatgaaatttgtggcgcagttgtcagtgtgcgtgggaagcaggaaagaattgctatttggacaaaaaatgctgccaatgaagccgctcaggtatatgcgctgtagtaacagtaacctcttcattggaaacacttgtaccataatttatttattttccttgctaacaaataccatgccttttctcataatattgagttactcaactaacatcttcattggaaacacttgtaccataccttatttagggcgtgtttggaagcaaagtatttcaaaaccatggttttagaatactatggttttgaaatgtcatgccacagctctagtattttttatatcactccaaacaatacttgatttgagacaaaatatctcaaaccataatatttagtatacatgtgcaagaagctagtcatagactttgggttggagtagagtttcaaatactccaaaagtaccatagtatctacaaaactatgttattcaaagcaaagtttgttacaaggtagccaaacacctcttggcaaaaaatgccatggtattctccaataccatggtttcacctcaaaactcaaaaatactttgcttccaaacacccccttattttccttgctaacaaataccatgccttttctcatattttgatcaagtttgagttattcacacataagcatgtattttattgctgtaaccttgccctgtggagcacttcgtaacatatgcatctgtcaaatcaatgtatatagatatttcagggcgtttcattttttttcagtattgttgttagttgttactgtctccttgcggaaccttccactcagctgttttagtcttttgagtaactggtgcaatatcttttgtaatatcaatctaaatatgtaagccggtcggaagccgctttttttgagccgctgcagctatgccacctcacatttgtactgtagaaaaaagcaggtgctactgggaacggcgtccttatctgttgacatttagacaatagttacttctattaaatcaaagtgtgctgggagtagtgccagggtattgccactactttccagtcattgctaatgctgggaactgaaaacctggtgggtattggcaacggtaaaatacatgtggtttttgaacttgttctctccttttcttacgttttcctttccttttcaaactgagagtaaacatccatcagtgcacgtatctcaaatctcagggtctgtttggtaacaatattttttcaaaactatagttttacaatactatacattacaattgtcatatgacaatatcatagttttggccagctccaagaatactagttattgtttggaactttggatacgacattgtaaaacatggtatttaggcaaaagctagccatgctataaaaactttaggttaggttggagtggagtttctaatacactacaaatatcatggttttgaaaatgcagttttcaggaatacatccaaacacctcatggtataaaatactacggtattcatataaaaaccataatattggctataactgcaaaaatacttttactttcaaacaggccctcgtgttactttcattactcaactggaaattcaatgggtattggcctattgggtagtgtggtgcaagtttcgaagaaaacatgtaaatgaaagggaaacatggatggtattggaccaacaaattggctatttccctcgtcctggccatgatacaggaaattgttgcgaggatcagtttttttaacccgtaccagtggattgttcttatgtcttagaaagttataaaatgttgcctcttcagcattgttgtcatttattccatatgtgtaataactaattagactaaacatgccgagcattttcctcctctatcaggtaagcatcgggaagcagtggaaggaacttctggactacaaggactccattggattcattgttcatgtgagcagggcttaagcctccctttgttaatgtcttgtttagtaagctgcaccatttctgactttgcccttttgtgctgcaggacgatgcaaagaagatggacaaaggcctgaagaaccgttacacggtttgatcctgatgctggaatcccagagagttgtagcagtagctggcccaaatgcatgctgggtggttttatctattgctgatgagttgtttaagcaagatccattcctgtattgtactccataagccacttgggtgttgctttgtattgttcactgctgtgttttttctgtggcaccgcatctacagtactagttgcttgtcgttgttcagttgcgcttgaatctgaactaacggagtctgagcaaaacacagcatattcaaacacggcgagcagcaccgtagccaaggcgacaggaactcaggaagtgcggtgaattagatttgttagatataatcaagttgaatttgtatttagactctgatgggcctgtttgtttcagattatataattaggcttaaataatctagatgagaaacaaacagataaattattaagagataaattattaagggggcgtttgaatgcactatacttatagttagttggctaaaaattgttagtggaattagctagctaataaataactaccgaactattaactaatttaccaacaatagctaatagctgaactattagttagggtgtttggatgtttccactaattttagccacgaactattatctcttgtgcattcaaacaccatctaagttagattattgtcgggtaccgtaattaggggtatccccaacactcctaaacacggctggtaaccaccatcattacaagctacagagactgatgggcacgattcaggtcaaggcttcgtctatccaagggacgcgatctcgcctcgtccgagcccagcctcgggcgggaactgtagtcccgaacgaagttacgtctcgcccaagggcctcctcaggcagtgggcgcaccctcggcacgcccgaggcccaactcgggcaagcttcccaactcgggcaagcttcgtcgagaagcaaccttggccaaatcgcctctccaaccgaccgtatcgcagtcgcattcaatacgaggatcgtctgacaccttatcctgacacacgtgcctcagtcggcaggatcgaagtgaccgcagtcacttcaccctttcgctgaccgacctgacaggaaaacagcgccgctcgccccgctccgactactgtaccacccgccagggtgaggctgacaacagccaagttcagcctcgggcgcaacaggaagctccgccttgcccgaccccagggctcggtctcaacctcgaccttggaaggtggtctccgcctcgcccgaccccagggctcggcctcaacctcggcctcggaaggtggtctccgcctcgcccgatcccagggctcggcttcaacctcggcctcgggagga

SEQ ID NO: 10 - DNA, 6996 bp, Zea mays
  source 1..6996: mol_type = genomic DNA; note = eif4e2 genomic sequence with 3' and 5' UTR
gcaactgctgtataattctacccaaatatctgttaaaaaatgtaaacaaaatgattgcaactgctggtgtgttgtaattgcaactgctggtgtggcgtggatgcaactgctgagttactctgatgtgattgcaagtactgaataactctgggaaattttgttaaaaaaatatgattgcaactgttggtctggtgtaattgcaactgctggtctggtgtgattgcaacttctatataactatgtccaaaatctgttaaacaaataatgattgcaactgctgtctggtgtaattgcaactactggtctggtgtgattgcaacttctatgtaactatatccaaaaatctgttaaacaaacaatgattgcaactactgtctggtgtaattgcaactgctgatctggtgtgattgcaacttctatataactatgtccaaaaatctgttaaacaaataatgattgcaactgctgtctggtgtaattgcaactgctggtctggtgtgattgcaacctctatataactatgtccaaaaatctgttaaaacaaacaatgattgcaactgttgtctggtaattgcaactgctggtctagtgtgattgcaacttctatataactatgtccaaaaatttgttaaacaaaacaatgattgcaactgttgtctggtataattgcaactgctgatctggtgtgattgcaacttatttgaagaggttgagccgttcgcggaggagccgttcacggagaaagaggggcggaggcgaggaagtaaaaaagttctgtcagaagcgtgcgggagggacgaagacggcctgggtggggcggttagctcggacctggcgcgtaattcggcctgggtgcattctctatattgaatgcacccaggtgtaatatataaaaacagtgtatatatatatatatatatatatatatatatatatatatatatatatatatatatatatatatatatatatatatatatatatatatatatatatatatatataatatatatatatatatatatatatatatatatatatatatatatatatatatatatatatatatatatatatatatatacacacacacacacacacacaaagctgagacaaaaacaacacctaggcacctaggtgactgacctatggaacatacaggaatacattcgacatcatgttagattatgcttaagcaactcctcagcaaatagagaaccttcaacacactctgcaaggtgctacggaatcggaaagaaccaccctacatgctactactctctgggatcaaaccgtgtaacggttcttcagtcccttgtccatcttctttgcatcgtcctgcagccaaagagggccaagtgagagtaggcacgccttatgagaggaggggtggcttaagcaatgagcactggtactcacatgaacaatgaatccaatggagtccttgtaatccagaaattccttccactgcttcccgatgcttatctgaaaggaaggggggacatgttcagaaccttatgtcaagcatgtcaggaggatgctggcatccaaatgaacttacataagccccccccccccccccccccccccccccaaaaaaaatactgtaaagcatggtatcaaaaaaacaattctcaacatagcaccatggaaatggccaactaccagccggatccccaaccaacaccccccttctgttttcctcgttattctttcgtatatcagggttacatcgataacacagcacgcttatatggaataataactcaaacctgacaatttgcatgtatgataatattggaattgtatcagggaaaaagtatattatttgtaaacatggaaaatatataggttgttgtacaagtattcatcgtgaaaccgaaaactatcataaaagtgtgatgattgcataggagaaaggaaacaagctgtttttaaaagcaacttagaaagagatgcgactgcaggctgcagcacccatacgaccgtacctgagcagcttcattagcagcattttttgtccagatagctattctttcctgcttgctacgcacgctgactactgctccacaaatttcatcaccatagtcgaattgttcgccgatcatagccaacagctacaagaaacagtgtgtaagggtggaggactggaggctataataaggacagatgtcaagaacaaaactcccttttctgataaaataaaactgcttacggtgtgcagccacagcgtgtcagattttcctcttccacagctgatggtccatttaccgccattagcacaaatagggtcttcccattttggctcaatcttgttcttgaagcaatggaagtcagctccaccgatcaacttgctagggtgatggatattattgtagaggctgcgaaatcacagatccattagatggaatatatttatagacgaaagccaaagttggacggggacacaaaaggaccaaaccaaagatagatcagcacatgtctgacacgggattagaccaagagcacttatcacaaggaagagaggaagggttattaaatatcagaccttccaactttcgatcacgcaccacaaccgttaagaaactcgacaggaagtcaagcaagagagccaataacctatgaagctcagttcatcagatcaaatcgaatctcgggttctgaatataaaaaaatatacattacagattttgcgtcatgacagagattgaattttctcatataaactagatccatgaggcgggtgtcactttgacttaataccaactgtagctgtagacaggggcgaatctagggctagggttgaagccccagtcgcagcccaatagcacctatactataccatcattttagcccacaaagtccaactaaaataggtcacaacctccttcctggccgcgccattcagcacctgcgtccggcctccgtccacgccgctagtaccagcgcggcagcgcgggcatccacagcagctagcagtgtggatggccgacacacctaaagcagctacagcagctagcagcgcacaggtgctcagcgaccaatgcaaggcacaagctgcaggcaggctgctgcagcacgccaagcctgcagcacaccgctggctgcccctaagacgtccgccgcatacgctggcgtctccgtcaagtgtgccttatagcccctcttttgatccattcctggatccaccactgactgtagacttgtcaaggcgtgtgtttgagtatgaacaaggactagcaggtaggtctggagatgtttgagaatggaaacataccatgcttacccaataaagatttcatacagtatgtgcatgaatcaaaccgaaagaggtgtaagattgatcgacagctgcttctaaaaaggcaaatgagaaggctaatatctatgatatagcaggggcaaaaacatgtaaaaagtgcagagacagctcgcaacatcagaacaacaggtttgcgtggtttcggggataaatctctaaatggctagaataactaggtgagcaaaaaacatgtgaaaatgaatatctgcttcggataggcgccttgcaaatgaagctaagacagtaactatgctctcagaattgatctttcccgctgtataaacaagtgttagaagctgaatgagagtgctatggattaggcatagtcagaaactaatgcgttaattaatctagagatccaagatccgtaaaccatggcagatttggctcgtatattgtgaatctggacttgtaccagtagttgtgagagtgtgagaggagggaaaggaggttaagaggtgtgcatgtgtcagagagagatagtcggacgcacccccagaagtcctctacggtggagaaggtgtggatgggtcggatggagctcccccaggcggccatcttgttcttgccgtgcgggttgtcgaaccagaaggtccacgagtgctcgagcgggtgcgcggcggggagggcgggcgagggggcgtcggcggcgatctcgccctcctcccggtcgtcgtcctcggtagcgtgcgcaggacggccgcgggagcccgcggaggccggcctcgtgtcaatctcgtcggccatggcagcggcggcggatggggatgggtgcggcgagtcgtgtccaaggaggtttggccgtttggggctcggggctggctgtctggtttttattttttcctttttcttttttttaacggctgacagtctgcgggtctgcggtctttttttaagctaaacatggttccattgatcacttgtattatacacagttgttgttcttccaacttctgtgactactacgattttgtggtcgtcgaaatagtggcgcagttttcgagtactgaggagcaccacgtacaacaacttctagacctgtgggtatctgatcttgagggcccgatgacttcactgatgaaagatattggatgttgaaccggaggtgcgtgtccttcctccttccgctcaactaccaacatggtgcttaccacgtgagtcttgtatgagatatacagtagcagattctggaccggctggtcgctggtggtccggttaagcggcttgaggacgggtgggttgttaggaactttttgagatcctcgaatgctccttgggtatcaggagtccactagaacctgtccaccttttgagcagcttgtacaatagcatgcctctttcgtcgagccatgaaataaaatgactcagcgctgccatgcatccagtaagcttctggaccttttgcgatcgtgatggctccatccgcagtatggattcaatcttcttagggttggcttcaatcccccgatagctgatgatgaaaccaagtatcgaacacacacttgttggggttaagctttcaacggtagcgcctcaggctattgaaaaacttgttggatgtcgtcaatgaagttttctggatcctttgtcttgattacaacatcatcgacataggcttccacacgtttgccccagtggtcggctaagtatgtttggatggcccgctggtaggtcgccctagcattctctaagccgaacgacatggaggtgtaacaaaacaccctgaagggtgtgatgaacgacgtcttctcctgaatcctcctcttgattctcctccactagacccatctggtggtatcccgagtaacagtccaggaaggagagcatggaacaccccgcggtagagttgactacttggtctattctagggagcccgaagggatccttcggatagtgtttgttgagatcagtatagtcgacgcacatgcgccaataaactttattccttttgagtacaagaacaaaattcacaagccactcaagatccgtcacttctctattgaagctagcagctactaggcgagctaactcggctcgtatggcttttctcttatcaggtgtaaagcggcagcttctgtcggatatgttttgcttgcaggtacaccttcagtttgtgctcagtcagttccttgggaactccgggcatgtctgtgagatgccatgcagacacgtctcggttgtcctgcaggaaccggatgagcatgtcttcctatttgccggtgaggctggagctaatggtcgtcgtcttgcgtgcgtcaaggaggccaaggtcggccttcttggtttcttcaactaaccgtatggaagtggccaaggtttcttctttcgccaggatcatgaggtcatcccccggtgatgtgtcagcgttcgtcgtcggtgttcgtggtgccgccaaggggccgatggtgagtgtcatttggatggcccccgggtagcactctgctgctccttggaagtcagcatggatggtgatgacaccctagggctcgggcatcttgagcaccatataagggtagtgggggatggccatgaacttggccagccccggcctcccgttgatggtgttgtagccgcactcgaaccgagccacctcaaagcgtaggaactcagttcggtagttatcaagggtgccaaaggtcacgacatgtagaggtggctgtgggggacagatattccccgggtccactagaaggttggaaggcctcgcgaaagactctaggcccattatttcgcaaggccaccccttcgtgggccaggggaggaatcactggaagagtggatcgacgcaagattggatcaactcgagcccaggcagcccaatggatttaaacgttatccgtagctttaatctgattctcccgcgcagcaccctcagacgttggaactgaacgaggataagtcggcaggataatagggagataagttcagtcggctcactattacttaggagcacatagttatcatacccgcatgtaacgcctcacggtcgagtatataaggcctagggggcaccccatcaagagacatgtcactcattagccatctacactattctctggcatccttcatactagagaatccccctgtaacccaccacataaagatccacaccaggaagtagggtattacacctctctaagcggcccgaacctgtagaaaattgtctatcgtctctcgtgcacctagcacgaaccatcgagctacagtcggttacaccgtcctacccaaaagcacctcgaggggtaacctcaggtgcgcggttgggtcccaaacccaacagctggtgcgccaggtagggggtgtgtcactgatccaagttagctcaatggccatcactcttcagcacaagactgctctccgccccggattcgtgttctgcttcggaaccatctcgttcgtggcagacaaagaagggactctacatcgtatagcggatctgccagagaaaaatccttccttgaaaatctctgagaaagctgaaacaaagcagaaaatagcgcaacctccagcgctccgagcgaagattgcctcctgtagaccaagagcagagatttcattcacccggagaaccccgttgtccacatcatccatagaaaaatggacacagattacaaggaaaaaaaagaaacaaagacccatcaagctgcccttctggtacctctgccctcaaagga

SEQ ID NO: 11 - DNA, 9304 bp, Zea mays
  source 1..9304: mol_type = genomic DNA; note = apb1 genomic sequence 5' and 3'UTR
gaagaggtactctttcaatcatagcaagatagtcacccctgggtcggcgtattcccgccaccttgaagggtccttcccagttgggggagagttgcagagcccttcttggtttggtacttgccgtaggactaggtcccgaccctaggttgccatctgcgtttgatgagggagtcctcgtcctcgtgctgtagccatttctgcatggacctgtcagaagtctagacccatggggagtccagaagggtttccgggggaaggcaggcttcggccccatagaccggggagcacggggtccccctgggggtccatctagttgtccccacctaagtaggagcctctagcgaagacatccttcaggtccccctcgggtgactgatactcgaggtcccgttcagctacgaccacctcaccgtcgtcgaccttttctttgccaggtcggcgatgaggtggggagccgtccctggaagactgctcgcgccgctcgctgacgcatttcgcgaggtcaatgatcttgcgacactctgcggcgttgtggcgaccgttggggtgcacagggcatgagccgctgttacccctctgcgaccgtgggcgtttgttgcggtcgccccggcccccggtcgcggctgcaacaactagagcgatggaccgcgacctctggtagtcgcggcccttcttctttttcttcttaccgtcccgggggacggcacccgagccacccgactgggcagccccggtttgtggagccgagtgccatgcacggccctcggcggctctggcgcttttgtcggccagagcgaagagtgtggggacagtctccacgtcatgcgtggccaacttctccaacattttctcatcacgtactccctgtcggaaggccgtgatgatggaagcatcggaaatgcaaggtatagtacctcgcaccttggtgaagcgggagatgaacgtccggagagtctccccgggctcctgcctcaccgcatggaggtgggcctccacaccgtgctgctggtaagcgctggcgaagtttgcaacgaaccgcgcgcagagctcttcctaggagtagattgaccctggtgcgaggttcatgagccaagtccaggcaggaccagacaaggcgacatgaaaatatgttgccatcacagcggtgtttccaccggctgctatgatagcggtgacatacacctgcaggaacttcgacggatttgacgttccgtcgtacttttccggcaggtgcggccggaacttgggtggccatgacgccgcacggagatgatccgcgagagcggcgcagtccacatcggccaaggggacacccgcttgggatcgggtacccattggggcctgcggtgctaccgcatcgaagtcttgattgaggttgcagccctcgatgttttggcggcgctcgcgcgccctttccaaagagacccgagcgtcctctcctgcacgcctgcgattgagttctgcccgaaggtcgttggtttgtgcacccctcactgagggtgagtgcacagacgtagacgcctcatgttggcgccgggatgacggtggcctcgacctggttgaggtagaatgtgccatgccgagcagtcggtcgacgtcatcccgccactgcttcatggccctcagtgaggccgtggagcttggagggtgtcgcagcaactccctggctactgacaacgctcccggagccgccctcgacggagttcaggatgtctgcgcagctgtatgctgccgtgcagcgtgcacagcagcagtgctcgacactgggcagttgggaacctgtggtgtggaagaaacaacttcttcctccacaaggaaatccgttgatgtctcggcgcggtgctccacgatttgcaccatcatgctgagcgaggatgcaagcaaaaacctaatgcctagccccctacctggcgcgccaaatgtcggagagggaattctccggctgggtggcggagtgcacctgccctaaatcctaagatgaggaaggggcttaagcgttttgcctgtcctgctaagcgaacatcaagcacatgaacacgcgagagtttagagtggttcgggctgccggagcgtaacaccctactccactgtatgttggattgctgtggaagcttgagaattgagagtctaagttgggtctaaaaattgagcttgtgttgtaacgttgcatgcctccccttttatagctaaaggggggcatgtacaagggtactgagcctcgacatgtgggcccaggaacatagcggatgcagtacttggagctactaatatttgctgctggggcaatcttcttgtgccctgacacctgagatctgcgtagcctcggcgtgcaggggaagcttctcgtataagggatgatgagcacagtgcaccgtgcagcacggtcgcactgtttcgacaatggactggataggtgagccgtctgcagggtggccctggcgccgcctgccagcggagtggacatgacgcaataaatgctgagggggcacatcacctgtcagcagggtggacaggcggcgcgtcctttccgtaataaatgcagagtccacgcggtctaggggccttacgtcaggctccgcccgctggcttattcacgggcaacgagccacgtggccgcatcgggtctccgcctgagtggggagcgcatgcgcagagtaaggcccgaccacgtgttagtaccggacccttgctcatgccagggtttcccctgtcccgggacctcgttgcggcccagacctttctcgaagagatctggaccccatccaagggacccgacatgcttactcgggagtcccgggccataatcgggggtccaggttgtgcgtacaggggtccgactcgactgatacatcctggatgtatcatctttccttgccacatggtgccccttgagctgcccatgtggtggggtcgagcgctgttcaccgtgtggctagggaacggcgtacgggcaccgcgtcttcatactgtagtaaggggtacccctgattcagggtaccgacaataattatgtaaactccttgagtaattggtgtctcatgtgtgatttctccatggcgttgcttcataaggatcacaaacatatttgtctcaccttttgaagcaaacataaatcaaaccctgtgacttgtaccatatcaccatatatgagttcaaatcatggcttcaagtcaccttactgatgcatcaacatgttataactcttcataactgattagttcaccgacttagtgcaagtactctcttcttcaccttaaccatggtacttcggtccacaagccgtcgcttggccttcaccttcgcttagttcctcgaagccctttccttgctatcttcaccctatcaaaccatctttgagtcacattatattgatcatccattgagagaatcatttcttcgatattgtgaaccttgcttgaatgtcatctagatatgctaagatcaatcaagctctagtttgattctcataaaagcatatatggactaatagtaatatggccaagccaattcacgatttctcatatcttattcactttggcttgaccaatcctcttaattacttcaacctctattatgatcatatttatgcatagtggtttctaaggtcttgtccatatattcaaaccaatatagagaccgtattatatccatttgcattgtctcactggttatttaaccttgtgttgaacctttattcactgatcattatacactattcaaatatgttcatcatactgaattttctataatataaaatagtgttttgcacgaccataaatggttagagacgttataccgttcttgttgccttgtttatgtcctagcaagattttcgttgataagagaggagagccataaacaggtcgtccggcctggcaagccgaggtttgtcctgcgtggtgcggcttgtctctgggccggctgctcccacaagctatctgagccgctgctccatttgatcggacagtcgacaccgtccgagcggcgatcaaaaccaccttcgacgactacgccgaaaccctagcctctatctcattccactccgacattcacgtgcagctgtcgggagcaggcaatggcgccggatctaagcgaactcgccgccgccgctccagcccgtggcgcctacctcgccggcgtcggtgtcgcggtcctcctcgctgcctccttcctcccagtagccgagtcgtcctgcgtgcgaggtgcaccctcaccctgccgagctttttgccactgagaccagcactgctaagtgctagtggggtttgggcttaactggcgtgttaaatagcaggctgggtcttgttttggtcgttagggagtgggggaaggttgaggggaattgggcttagtggctcggtttgcattgggactaattggggtagtttaggttaacaatcaaattagggtggtttggtgtgggctccaatagtttcagcccatgggccccccaagcccgcgcctccgcctccgcctccgctgccggccgtcgcaacgactccaactccacccaagtccactacaggcctacagcctactcaccgggttactactacagttcgcaactacatccagattccaggctaatgctaatccttaggttggagatgggttggaaacagattgaacttgcggtgacataaatggattggtttggtttggcttggattctgcaagttcacacgcttaattcaaagtatatctgggtggattcagattgatttacagcacaacaattcccatctgccacctcaaagaatggattcaccatttgccacttcgtttttttttactgccattggacatgcgaaatggccatgctgcccataatcctaattgattatgtgtagttagcactcattgctcttcaacctgcaaaaatcaagcatagtgccatgctgtagtttgccgctctagatctacatgaaatggatcccaacccaaaatacagcctatcatctcaattcacgcatagccaccactcaagcaaagtaaattctgaacttgtttttaagtggccaaatcgatctccgtgagacccattacaacagcaaccaacttattttgactcacttggagcacaacggtagtaggggttgggctgcgtgctggagcagcagtggtagtagtggtgtcttaggccttctgcttctgccaagctcaactctgcacccaagaaggtgactgggtctgactgggaggagaagcagttgtaatagtcgcttttgcagtcttctgcttctgccgagctcaacttgccaccagagaagaaggttactgttctgtgggggagcatcgcagtctgctggtcatgccagctaagaacagagctgcatggcctgtctttggccgtcagagcgtggaagcactgaaggttctggcagcattgacttctgtgtcatcatcgtacagtggtagaaaaactaagggaagttgaatggaccaaacattaagatcttggtcaattggggtaaggggcaatatcgactaaaaagagtaaattagcaggaaaagcaaattggcaatgttaaacatggcagatggggagatctgatttcgtaaaaaaactctccctccctttcccctccctcttcattctcttctcgaaattcatgaaacttctaaaattgttgtttgaccttacccacaattttggtgtgaatgagcagtatttctggatataataattcgactggggcagttacatccccccctccctcaaacacacaatattcacaatagcagtgaccatatttctttttctacaattgtagcagatctgattagtgtatctgatattattatgtatacagataactcattggtgagagacataagccaaatgccgcaaagcagctatgggattgaaggattgtcacatataacagttgctggtgcgctcaatcatgggatgaaggaggtaatgataaaccctagggccacatcgtgtttcccaaacaatatgggaatgaacaattgaagtcaaagataagttgaatgtaatcgtgtattttcatgtcttgtatgtgcaggtctggtttctgtggacactgtagatagttactgaaccttgatgcatgatagtttttcacttctatttgaactaaaatttggactgtaacgttcaattggtgtgctatgaaatcaaagtacttaattatgtgtagatatgtttgatagagtcatattcaaatagtctattgacagtgtagatactgttatgcttccttcttgcttcgtcatgaaatgttttcctgcaggtggaagtgtggcttcagacaataagtccaggtcaaaggacgccaatccacaggcattcctgtgaagaagttttcactgtcctcaaagggaagggtacgctcttgatgggatcaagctcactaaagtacccagggcagccacaggaaattcctttctttcagaataccacattttcaatccctgtaaatgatccacaccaggtaaaggaaatgagttattacttatatgcatttttactttttctgatacattagatgcctcatatcctggttatatttaactgtaacttggccagatttcaacatgtaaatttattttggtgggtaggaaattgatctttttgttaatgtatacatcatctttctcttgcaggtttggaattctgacgagcacgaagatttgcaagttcttgtgatcatatcgagaccgcctgctaagatgtgagcatgcctaactttaacatcgtgtttcattttttttctcaaacatgtaatagagctgcacatcattatattaagcagaggaaaaagagatgagtcttaacaactcatacaacccttaaggccatgttcggttctaccccaatccatatggattggattgagggggtttcaatccctagtaagtcaaaatctctccaaatccgtatcaatcccctccaatctatatggattgaaaataaccgaacaagccctaaaggggcaaacaccgcactaggacttaccacacactaatacacaaccaacaccaaggaggaggaatcatggccatagactacagagttgtttttatatttctcattatgctatcaaaactaggtgactccctcaaacacatgcatttctatgtttccagatccaccaagccactaaaactaacaacgagtttaagcccttacgagattgcttagctgctgtgcagacatcaccattcagtaaagatgttgtcccttaacccctgaacatactgctgcagcccaaacaactttagggcagactacaccacacagtccttgcaaatacacaggaggtgagcaaatgttgtatgttctcctcttcttgatcacacaatggacagctctcatgatgctcaagtccccagtgagctaacccgtctgcagtccaacactgatttagagcagctagccaaatgaaaaacttgcatcggaggtgcagcccaaaacttccggatttctttccatgggtcaaaaccaacagccccgaacacccgacaaagaaactacaataagcagatttggttgagaattctcctgtagtggatggtgatcaaatgtgccagtaggtcgtgtattccctggtgaagatcaatttctgctataatatcccatagatggtggtattccatcaaggctgtagctgacagccccctgatgtaccatacccaagcattattgtccaatgcatcctgcaccattcttcttttgattgctctctttgccacatgtgtgtaaagtggagcgggatcaaacgcagatattccctgaaaccatttatcactccaaaaaagttttcggctgtcattgcctaacacagaataagctgaaatgcagaacatggacatagaattacagtgaaccttgtagagagtattcccttatgtttccatcttgttcaactcagtaccatactaagttgacaagacttgattggagaatttgctacctatactgctgtgatttattgatttaccatttttttctgtaattgtccaacattgctagaatagatatcatagtttcatgcacgtgtcatgggcacttttgttatctatgtcagtagcatcttttatgctctagtgttgaccctagcaatattgataatacagcaactgagtaatccacatacgcgtttcttttctccccctgtttgcaattgcatgtcagaaaacgcacataatcatatctgcaatcaacatttttctttctgagatccaatgacatcctcgttgcagatttttatatgatgattggagcatgcctcatacagccgcggtactgaaattccccttcgtctgggatgaggactgcttcgaagcagcaaaagacgaactctaggtcacaagtgtttcctgcaatttatctgcttcatccatgatcctgctggtgctggactactacaattctcagcactagttgtaataaagccagtgcgcttttcatgtataattctgtattgtggctcgcgaaaataaaatttggcaacggtttatgaacttatgcgcttgtggagttgatccaatgttcaccgtttcaagatttttgcattattactgtgctccaggtttcgagcagaagcagtactgccttcatccatgaacttggggtttgtttgatagcccccgtaacagatttcgggcagaatttaggagcttggaaacagtcttagtatgaaaccgagagtggtttctaggatgtaattggatcgaatacggacggatatcactgaaactatatttgttttcatatttttatttggatttgaatttgaatacatatggccttggatacaaatataaatatggccgatgcaaatattgataatcataagtcgaatacggaattaatcgaacacagataacgtcgggtagcagattttttgtcagatatcatataaagaccgttccacacatatcatggttattgtaatcattagtcactttttgagtgacattttattttttaggattatatttggacatatattgtggctctataaaattttatgaatttctaagtatttgagtattattaattttttatgcagaaaatcattaaaatacatttaaatttataaaatattctaatgtcgatcgaaaattgcaaataaattaccaagactagcaaacattctataaaaagataatctcaagtcttcacatgaaaatgataaagtgaaatattgattatgtcgtaacttggatacttagaatgatttcacgtgtaactcatgcaacaagtggaagtgaaatgaaaaaaatgttggtatcttgtggatcttatggcccaaattttatgaggattata

SEQ ID NO: 12 - protein, 201 aa, Zea mays
  source 1..201: note = APB1
MAPDLSELAAAAPARGAYLAGVGVAVLLAASFLPVAESSCVRDNSLVRDISQMPQSSYGIEGLSHITVAGALNHGMKEVEVWLQTISPGQRTPIHRHSCEEVFTVLKGKGTLLMGSSSLKYPGQPQEIPFFQNTTFSIPVNDPHQVWNSDEHEDLQVLVIISRPPAKIFLYDDWSMPHTAAVLKFPFVWDEDCFEAAKDEL

SEQ ID NO: 13 - protein, 84 aa, synthetic construct
  source 1..84: note = AlleleE1_a_short MutE1_9_16
MAEETEAAPRPRTTTGRRARSPTSPALLPRPPRTRSSTPGPSGSTTRRASPSRPPGGAPSAPYTPSPPSRNSGGFTITLITLAN

SEQ ID NO: 14 - protein, 74 aa, synthetic construct
  source 1..74: note = AlleleE1_b_short MutE1_10_30_
MAEETDTTRGRRQGGGRDHRPRLRSFPARHAPARALLDLLVRQPAEQVQAGRLGELHPPHTHLLHRRGILGALQ

SEQ ID NO: 15 - protein, 86 aa, synthetic construct
  source 1..86: note = AlleleE1_c_short MutE1_15_19_
MAEETDTRPAFRGLPRPPRARGRRQGGGRDHRPRLRSFPARHAPARALLDLLVRQPAEQVQAGRLGELHPPHTHLLHRRGILGALQ

SEQ ID NO: 16 - protein, 198 aa, synthetic construct
  source 1..198: note = AlleleE2_a MutE2_14_15 / 56_66
MADEIDTRPASAGSRGRPAHAADAPSPALPAAHPLEHSWTFWFDNPHGKNKMAAWGSSIRPIHTFSTVEDFWGLYNNIHHPSKLIGGADFHCFKNKIELGRPYLCCGRGKSDTLWLHTLLAMIGEQFDYGDEICGAVVSVRSKQERIAIWTKNAANEAAQISIGKQWKEFLDYKDSIGFIVHDDAKKMDKGLKNRYTV

SEQ ID NO: 17 - protein, 80 aa, synthetic construct
  source 1..80: note = AlleleE2_b_short MutE2_58_77
MADEIDTRPASAGSRGRRQRGGRDRRRRPLARPPRRAPARALVDLLVRQPARQKQDGRLGELHPTHSHLFHRRRLLGPLQ

SEQ ID NO: 18 - protein, 88 aa, synthetic construct
  source 1..88: note = AlleleE2_c_short MutE2_59_83 / 59_74
MADEIDTRPASAGSRGRPAHANRGRRQRGGRDRRRRPLARPPRRAPARALVDLLVRQPARQKQDGRLGELHPTHSHLFHRRRLLGPLQ

SEQ ID NO: 19 - DNA, 23 bp, synthetic construct
  source 1..23: mol_type = other DNA; note = gRNA14 / ZmeIF4E_a_SpCas9_gRNA14 / Exon1 of eIF4E1
cgacaccaggcctgcctccgcgg

SEQ ID NO: 20 - DNA, 22 bp, synthetic construct
  source 1..22: mol_type = other DNA; note = gRNA47 / ZmeIF4E_b_SpCas9_gRNA47
ccgtcctgcgcacgctaccgag

SEQ ID NO: 21 - DNA, 23 bp, synthetic construct
  source 1..23: mol_type = other DNA; note = ZmeIF4E_a324&b330_SpCas9_gRNA
caaatagggtcttcccattttgg

SEQ ID NO: 22 - DNA, 23 bp, synthetic construct
  source 1..23: mol_type = other DNA; note = ZmeIF4E_a367&b373_SpCas9
ttttcctcttccacagctgatgg

SEQ ID NO: 23 - DNA, 4206 bp, synthetic construct
  source 1..4206: mol_type = other DNA; note = V40 NLS-SpCas9-Nucleoplasmin NLS, maize optimised
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatcatatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaagcgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggacaagaggccggcggcaaccaagaaggcgggccaggccaagaagaagaagtga

SEQ ID NO: 24 - DNA, 572 bp, Zea mays
  source 1..572: mol_type = genomic DNA; note = ZmU6 promotor
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgtttt

SEQ ID NO: 25 - DNA, 1992 bp, Zea mays
  source 1..1992: mol_type = genomic DNA; note = ZmUbiquitin promoter+ 5'UTR
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 26 - DNA, 177 bp, synthetic construct
  source 1..177: mol_type = other DNA
gcggatggagctcccccagggtttcagagctatgctggaaacagcatagcaagttgaaataaggctagtccgttatcaacttgaaaaagtggcaccgagtcggtgcagtccaagcaggatgattgggggagctctctctccgcggttctatctagttacgcgttaaaccaactagaa

SEQ ID NO: 27 - DNA, 181 bp, synthetic construct
  source 1..181: mol_type = other DNA
ggggcggatggagctcccccgtttcagagctatgctggaaacagcatagcaagttgaaataaggctagtccgttatcaacttgaaaaagtggcaccgagtcggtgcagtccaagcaggatgattgggggagctccatctctctccgcggttctatctagttacgcgttaaaccaactagaa

SEQ ID NO: 28 - DNA, 185 bp, synthetic construct
  source 1..185: mol_type = other DNA
gcccgcagagcaagtccaagcgtttcagagctatgctggaaacagcatagcaagttgaaataaggctagtccgttatcaacttgaaaaagtggcaccgagtcggtgcggagctcccccaatcatcctgcttggacttgcttctctctccgcggttctatctagttacgcgttaaaccaactagaa

SEQ ID NO: 29 - DNA, 177 bp, synthetic construct
  source 1..177: mol_type = other DNA
gtcggatggagctcccccagggtttcagagctatgctggaaacagcatagcaagttgaaataaggctagtccgttatcaacttgaaaaagtggcaccgagtcggtgcaaacaagatggatgattgggggagctctctctccgcggttctatctagttacgcgttaaaccaactagaa

SEQ ID NO: 30 - DNA, 180 bp, synthetic construct
  source 1..180: mol_type = other DNA
gggtcggatggagctcccccgtttcagagctatgctggaaacagcatagcaagttgaaataaggctagtccgttatcaacttgaaaaagtggcaccgagtcggtgcaaacaagatggatgattgggggagctccatctctctccgcggttctatctagttacgcgttaaaccaactagaa

SEQ ID NO: 31 - DNA, 186 bp, synthetic construct
  source 1..186: mol_type = other DNA
gcccgcacggcaaaaacaagagtttcagagctatgctggaaacagcatagcaagttgaaataaggctagtccgttatcaacttgaaaaagtggcaccgagtcggtgcggagctcccccaatcatccatcttgtttttgccgtctctctccgcggttctatctagttacgcgttaaaccaactagaa

SEQ ID NO: 32 - DNA, 187 bp, synthetic construct
  source 1..187: mol_type = other DNA
gcgatcttgttcttgaagcaagtttcagagctatgctggaaacagcatagcaagttgaaataaggctagtccgttatcaacttgaaaaagtggcaccgagtcggtgcgatcgtgggagccggattccattgcttcaagaacatctctctccgcggttctatctagttacgcgttaaaccaactagaa

SEQ ID NO: 33 - DNA, 188 bp, synthetic construct
  source 1..188: mol_type = other DNA
gcaccctagcaaattgatcgtgtttcagagctatgctggaaacagcatagcaagttgaaataaggctagtccgttatcaacttgaaaaagtggcaccgagtcggtgcaagcaatggaatccggctcccacgatcaatttgctatctctctccgcggttctatctagttacgcgttaaaccaactagaa

SEQ ID NO: 34 - DNA, 189 bp, synthetic construct
  source 1..189: mol_type = other DNA
gtcaccctagcaaattgatcggtttcagagctatgctggaaacagcatagcaagttgaaataaggctagtccgttatcaacttgaaaaagtggcaccgagtcggtgcaagcaatggaatccggctcccacgatcaatttgctagtctctctccgcggttctatctagttacgcgttaaaccaactagaa

SEQ ID NO: 35 - DNA, 187 bp, synthetic construct
  source 1..187: mol_type = other DNA
gcaatcttgttcttgaagcaagtttcagagctatgctggaaacagcatagcaagttgaaataaggctagtccgttatcaacttgaaaaagtggcaccgagtcggtgcgatcggcggagccggattccattgcttcaagaacatctctctccgcggttctatctagttacgcgttaaaccaactagaa

SEQ ID NO: 36 - DNA, 187 bp, synthetic construct
  source 1..187: mol_type = other DNA
gcaccctagcaagttgatcgggtttcagagctatgctggaaacagcatagcaagttgaaataaggctagtccgttatcaacttgaaaaagtggcaccgagtcggtgcaagcaatggaatccggctccgccgatcaacttgcttctctctccgcggttctatctagttacgcgttaaaccaactagaa

SEQ ID NO: 37 - DNA, 190 bp, synthetic construct
  source 1..190: mol_type = other DNA
gcatcaccctagcaagttgatgtttcagagctatgctggaaacagcatagcaagttgaaataaggctagtccgttatcaacttgaaaaagtggcaccgagtcggtgcaagcaatggaatccggctccgccgatcaacttgctaggtctctctccgcggttctatctagttacgcgttaaaccaactagaa

SEQ ID NO: 38 - DNA, 188 bp, synthetic construct
  source 1..188: mol_type = other DNA
gcaccctagcaaattgatcgtgtttcagagctatgctggaaacagcatagcaagttgaaataaggctagtccgttatcaacttgaaaaagtggcaccgagtcggtgcaagcaatggaaaccggccctatcgatcaatttgctatctctctccgcggttctatctagttacgcgttaaaccaactagaa

SEQ ID NO: 39 - DNA, 189 bp, synthetic construct
  source 1..189: mol_type = other DNA
gtcaccctagcaaattgatcggtttcagagctatgctggaaacagcatagcaagttgaaataaggctagtccgttatcaacttgaaaaagtggcaccgagtcggtgcaagcaatggaaaccggccctatcgatcaatttgctagtctctctccgcggttctatctagttacgcgttaaaccaactagaa

SEQ ID NO: 40 - DNA, 192 bp, synthetic construct
  source 1..192: mol_type = other DNA
gcgatcttgttcttgaagcaagtttcagagctatgctggaaacagcatagcaagttgaaataaggctagtccgttatcaacttgaaaaagtggcaccgagtcggtgcaaattgatcgatagggccggtttccattgcttcaagaacatctctctccgcggttctatctagttacgcgttaaaccaactagaa

SEQ ID NO: 41 - DNA, 186 bp, synthetic construct
  source 1..186: mol_type = other DNA
gcaccctagcaagttgatcgggtttcagagctatgctggaaacagcatagcaagttgaaataaggctagtccgttatcaacttgaaaaagtggcaccgagtcggtgcagcaatggaagccggccctgtcgatcaacttgcttctctctccgcggttctatctagttacgcgttaaaccaactagaa

SEQ ID NO: 42 - DNA, 189 bp, synthetic construct
  source 1..189: mol_type = other DNA
gcatcaccctagcaagttgatgtttcagagctatgctggaaacagcatagcaagttgaaataaggctagtccgttatcaacttgaaaaagtggcaccgagtcggtgcagcaatggaagccggccctgtcgatcaacttgctaggtctctctccgcggttctatctagttacgcgttaaaccaactagaa

SEQ ID NO: 43 - DNA, 192 bp, synthetic construct
  source 1..192: mol_type = other DNA
gcaatcttgttcttgaagcaagtttcagagctatgctggaaacagcatagcaagttgaaataaggctagtccgttatcaacttgaaaaagtggcaccgagtcggtgcaagttgatcgacagggccggcttccattgcttcaagaacatctctctccgcggttctatctagttacgcgttaaaccaactagaa

SEQ ID NO: 44 - DNA, 182 bp, synthetic construct
  source 1..182: mol_type = other DNA
ggatgcaaagaagatggacaagtttcagagctatgctggaaacagcatagcaagttgaaataaggctagtccgttatcaacttgaaaaagtggcaccgagtcggtgcacggttcttcagcagcttgtccatcttctttctctctccgcggttctatctagttacgcgttaaaccaactagaa

SEQ ID NO: 45 - DNA, 189 bp, synthetic construct
  source 1..189: mol_type = other DNA
ggcaggacgatgcaaagaagagtttcagagctatgctggaaacagcatagcaagttgaaataaggctagtccgttatcaacttgaaaaagtggcaccgagtcggtgcacggttcttcagcagcttgtccatcttctttgcatcgtctctctccgcggttctatctagttacgcgttaaaccaactagaa

SEQ ID NO: 46 - DNA, 172 bp, synthetic construct
  source 1..172: mol_type = other DNA
gaaaccgtgtaacggttcttcgtttaagagctagaaatagcaagtttaaataaggctagtccgttatcaacttgaaaaagtggcaccgagtcggtgcaagatggacaagctgctgaagaaccgttactctctctccgcggttctatctagttacgcgttaaaccaactagaa

SEQ ID NO: 47 - DNA, 183 bp, synthetic construct
  source 1..183: mol_type = other DNA
gcgatgcaaagaagatggacagtttcagagctatgctggaaacagcatagcaagttgaaataaggctagtccgttatcaacttgaaaaagtggcaccgagtcggtgcacggttcttcagcagcttgtccatcttcttttctctctccgcggttctatctagttacgcgttaaaccaactagaa

(19) United States
(12) Patent Application Publication
(10) Pub. No.: US 2026/0116930 A1
(43) Pub. Date: Apr. 30, 2026

(54) eIF(ISO)4E PROTEIN VARIANTS FOR RESISTANCE TO MAIZE VIRAL DISEASES

(71) Applicant: LIMAGRAIN EUROPE, SAINT-BEAUZIRE (FR)

(72) Inventors: Christophe SALLAUD, BEAUMONT (FR); Stéphane LAFARGE, LA ROCHE BLANCHE (FR); Anna BASTET, SAINT CHRISTOL LES ALES (FR); Jean-Luc GALLOIS, MONTFAVET (FR); Céline VALEILLE, MONTFAVET (FR); Jacques ROUSTER, MIREFLEURS (FR)

(21) Appl. No.: 19/099,230

(22) PCT Filed: Jul. 27, 2023
(86) PCT No.: PCT/EP2023/070822

§ 371 Date: Jan. 28, 2025

(30) Foreign Application Priority Data
Jul. 29, 2022 (EP) 22306144.1
Jul. 29, 2022 (EP) 22306145.8

(51) Int. Cl. C07K 14/415; C12N 15/82

(52) U.S. Cl. CPC C07K 14/415; C12N 15/8283

(57) ABSTRACT

The present invention relates to an eIF(iso)4E protein variant able to confer resistance to at least one maize virus, in particular able to confer resistance to Maize Lethal Necrosis Disease (MLND). Said protein variant comprises at least one mutation by comparison to the corresponding native maize eIF(iso)4E protein. The present invention also relates to a nucleic acid encoding at least one eIF(iso)4E protein variant as defined above, a vector comprising said nucleic acid and a genetically modified or edited maize plant or seed resistant to at least one maize virus, comprising at least one eIF(iso)4E protein variant as defined above. The present invention also relates to a method for obtaining a genetically modified or edited maize plant resistant to at least one maize virus.

### FIELD OF THE INVENTION

The present invention relates to resistance of maize to viral diseases.

### BACKGROUND OF THE INVENTION

Maize can be affected by many viral diseases, in particular caused by several potyviruses, such as Sugarcane Mosaic Virus (SCMV), Maize Dwarf Mosaic Virus (MDMV), Wheat Streak Mosaic Virus (WSMV) and Maize Lethal Necrosis Disease (MLND) which results from the combination of two viruses: MCMV (Maize Chlorotic Mottle Virus) and one potyvirus of the SCMV or MDMV type. MCMV belongs the Machlomovirus genus in the Tombusviridae family.

Insecticide treatments are efficient in protecting the corn crop from MNLD and, until recently, little research has been conducted to find alternatives to these treatments. As insecticide regulations become more stringent in some parts of the world, this leads to increased insect pressure and, indirectly, an increase in plant viruses, as insects are important vectors of viruses.

Plant genetic resistance does exist to certain viruses in nature. Introgression of this genetic resistance in commercial varieties is an alternative to chemical treatments generating no impacts on the environment. Resistance to MLND viruses can be acquired using this natural variability, by introgression of QTLs known to confer a resistance so such diseases (Murithi et al., Frontiers in Genetics, 2021, 12, 767883:1-17) or by using favorable allele of genes known to be involved in resistance to SCMV for example (Leng et al., Molecular Plant, 2017, 10:1357-1359).

Natural resistance is not always available in certain crops. However, similar resistance can be created by mutagenesis, transgenesis or new breeding techniques.

eIF4E proteins have been used as target to improve genetic resistance of many vegetable crops. eIF4E proteins are indeed known to have a role in virus replication in plants. It was first demonstrated that eIF4E knock-out (KO) vegetable plants did confer a resistance to several viruses of the Potyviridae family. They have also been assessed in tomato (Gauffier et al., 2016, The Plant Journal, 85:717-729). Besides, point mutations able to disrupt interaction between some plant viruses and eIF4E proteins have been disclosed in vegetable plants, such as pepper (Charron et al., 2008, The Plant Journal, 54:56-58).

Therefore, there is a need to provide maize plants resistant to viral diseases, such as diseases caused by potyvirus and/or machlomovirus and other viruses able to infect maize, as an alternative to the use of insecticide treatments.

### DESCRIPTION OF THE INVENTION

The Inventors have now found new eIF4E and eIFiso4E protein variants able to confer virus resistance to maize, in particular resistance to at least one potyvirus and/or machlomovirus.

These eIF4E and eIFiso4E protein variants comprises at least one mutation by comparison to the corresponding native maize eIF4E and eIFiso4E protein.

Advantageously, eIF4E and eIFiso4E protein variants comprising at least two mutations present an increased potyvirus and/or machlomovirus resistance to maize.

Besides, to favor a long-term viral resistance, it is preferable to combine at least two mutations, preferably not located in the same region of the protein, so as to prevent the virus to circumvent the resistance.

The eIF4E and eIFiso4E protein variants according to the invention thus preferably combine one, two or at least three mutations, which are selected among new identified mutations and mutations already identified in eIF4E proteins of other plant species.

Efficient combinations of mutations comprise amino acid substitutions transposed from different plant species. The eIF4E and eIFiso4E protein variants of the invention do not result of a simple transposition of known eIF4E and eIFiso4E mutations of a single given species.

A first object of the inventions is an eIF(iso)4E protein variant able to confer resistance to at least one maize virus, wherein said protein variant comprises at least one mutation by comparison to the corresponding native maize eIF(iso)4E.

Said eIF(iso)4E protein variant is preferably an eIF4E1 protein variant, an eIF4E2 protein variant, an eIFiso4E1 protein variant or an eIFiso4E2 protein variant.

Said native maize eIF(iso)4E protein is for example protein eIF4E1 of sequence SEQ ID NO: 1, protein eIF4E2 of sequence SEQ ID NO: 2, protein eIFiso4E1 of sequence SEQ ID NO: 3 or protein eIFiso4E2 of sequence SEQ ID NO: 4.

Said maize virus is preferably a potyvirus or a machlomovirus.

Said at least one mutation preferably disrupts the interaction between said protein variant and at least one maize virus, preferably a virus selected from the group consisting of MCMV (Maize Chlorotic Mottle Virus), SCMV (Sugarcane Mosaic Virus), MDMV Maize (Dwarf Mosaic Virus) and WSMV (Wheat Streak Mosaic Virus).

Said at least one mutation may for example be selected from:
the group consisting of V96D, G97R, T7S, A19V, L331, N55D/K, P56S/T, A63D, A64D, S67A, D99G, S159K, Q164K and G211L/D/S, by reference to native maize protein of sequence SEQ ID NO: 1,
the group consisting of D101G, A65D, A66D, N57D/K, P58S/T, S69A, G98D, G99R, S161K, Q166K and G213L/D/S, by reference to native maize protein of sequence SEQ ID NO: 2,
the group consisting of A60D, A61D, N94R, W108L, S157K and R161E/L, by reference to native maize protein of sequence SEQ ID NO: 3, or
the group consisting of A54D, A55D, T88R, W102L, S151K and R155E/L, by reference to native maize protein of sequence SEQ ID NO: 4.

The protein variant as defined above preferably comprises at least two mutations by comparison to the corresponding native maize eIF(iso)4E protein.

The eIF(iso)4E protein variant as defined above may for example comprise:
mutations (i) A63D and A64D, (ii) V96D and G97R, (iii) D99G and/or (iv) G211L/D/S, by reference to native maize protein of sequence SEQ ID NO: 1,
mutations (i) A65D and A66D, (ii) G98D and G99R, (iii) D101G and/or (iv) G213L/D/S, by reference to native maize protein of sequence SEQ ID NO: 2,
mutations (i) A60D and A61D, (ii) N94R, (iii) W108L and R161E/L, and/or (iv) S157K, by reference to native maize protein of sequence SEQ ID NO: 3, or
mutations (i) A54D, A55D, (ii) T88R, (iii) W102L and R155E/L and/or (iv) S151K, by reference to native maize protein of sequence SEQ ID NO: 4.

The eIF(iso)4E protein variant as defined above for example comprises or consists of sequence SEQ ID NO: 5, SEQ ID NO: 6, SEQ ID NO: 7 or SEQ ID NO: 8.

Another object of the invention is a nucleic acid encoding at least one eIF(iso)4E protein variant as defined above.

Another object of the inventions is a vector comprising at least one nucleic acid encoding at least one eIF(iso)4E protein variant as defined above.

Another object of the invention is a genetically modified or edited maize plant resistant to at least one maize virus or a genetically modified or edited maize seed resistant to at least one maize virus, wherein said plant or seed comprises at least one eIF(iso)4E protein variant as defined above. Said plant is not obtained by means of an essentially biological process.

Another object of the invention is the progeny of a genetically modified or edited maize plant resistant to at least one maize virus, as defined above, or of a genetically modified or edited maize seed resistant to at least one maize virus, as defined above, wherein said plant or seed comprises at least one eIF(iso)4E protein variant as defined above.

Another object of the invention is a method for obtaining a genetically modified or edited maize plant resistant to at least one maize virus, wherein said method comprises:
transforming a maize cell or maize tissue with at least one vector as defined above, to obtain a transformed maize cell or a transformed maize tissue, and
regenerating a maize plant from the transformed maize cell or transformed maize tissue.

Another object of the invention is a method for obtaining an edited modified maize plant resistant to at least one maize virus, wherein said method comprises introducing at least one mutation in an endogenous maize gene encoding an eIF(iso)4E protein, to obtain a mutated gene encoding an eIF(iso)4E protein variant as defined above.

Another object of the invention is the use of at least one eIF(iso)4E protein variant as defined above for preventing at least one viral disease in maize, wherein said viral disease is preferably caused by one potyvirus and/or one machlomovirus.

Another object of the invention is a method for identifying a maize plant resistant to at least one maize virus, wherein said method comprises detecting in the cells of a maize plant the expression of an eIF(iso)4E protein variant as defined above and/or the presence of a nucleic acid encoding an eIF(iso)4E protein variant as defined above.

Native maize eIF(iso)4E proteins and eif4e genes

eIF(iso)4E genes code for eukaryotic translation initiation factor 4E (eIF4E) proteins and their isoforms.

The term “eIF(iso)4E” as used herein refers to eIF4E (eukaryotic translation initiation factor 4E) or its isoform eIFiso4E (Eukaryotic translation initiation factor isoform 4E).

There are two maize eIF4E proteins: protein eIF4E1, which is encoded by an eif4e1 and protein eIF4E2, which is encoded by an eif4e2 gene.

By “native maize protein”, it is herein meant a wild-type maize protein.

By “native maize gene”, it is herein meant a wild-type maize gene.

A reference sequence for native maize protein eIF4E1 is sequence SEQ ID NO: 1. Said sequence is also referred to as Zm00001d041682_eIF4E1 in B73 genome version 4 (identified as ZmA188v1aHC012309_eIF4E1 in J1A genome).

A reference sequence for native maize protein eIF4E2 is sequence SEQ ID NO: 2. Said sequence is also referred to as Zm00001d041973_eIF4E2 in B73 genome version 4, (identified as ZmA188v1aHC012641_eIF4E2 in J1A genome).

A reference sequence for native maize eif4e1 gene is sequence SEQ ID NO: 9.

The eif4e1 gene is located on chromosome 3, more precisely at positions chr3: 133075625 to 133080137 in B73 genome version 4. The eif4e1 gene comprises 5 exons. Exon 1 is located in chr3: 133075814 to 133076061 in strain B73. Exon 2 is located in chr3: 133077529 to 133077694 in strain B73.

A reference sequence for native maize an eif4e2 gene is sequence SEQ ID NO: 10.

The eif4e2 gene is located on chromosome 3, more precisely at positions chr3: 146285064 to 146288191 (reverse strand) in B73 genome version 4. The eif4e2 gene comprises 5 exons. Exon 1 is located on chr3: 146288074 to 146287821 in strain B73. Exon 2 is located chr3: 146286386 to 146286221 in strain B73 (reverse strand).

Maize comprises a single copy of the eif4e1 gene and a single copy of the eif4e2 gene.

The skilled person knows how to easily localize the eif4e1 and eif4e2 genes, as well as the corresponding exons 1 and 2, in a given maize strain, for example by sequence alignment with the sequences of said corresponding genes and exons in strain B73 as defined above.

There are two maize eIFiso4E proteins: protein eIFiso4E1 and eIFiso4E2.

A reference sequence for native maize protein eIFiso4E1 is sequence SEQ ID NO: 3.

A reference sequence for native maize protein eIFiso4E2 is sequence SEQ ID NO: 4.

eIF(Iso)4E Protein Variant

The present invention particularly relates to an eIF(iso)4E protein variant able to confer resistance to at least one virus able to infect maize.

The eIF(iso)4E protein variant may be an eIF4E1 protein variant, an eIF4E2 protein variant, an eIFiso4E1 protein variant or an eIFiso4E2 protein variant.

By “maize virus”, it is herein meant a virus able to infect maize.

The maize virus as defined above is preferably a potyvirus or a machlomovirus.

The potyvirus may for example be selected from the group consisting of Sugarcane Mosaic Virus (SCMV), Maize Dwarf Mosaic Virus (MDMV) and Wheat Streak Mosaic Virus (WSMV).

The machlomovirus is for example MCMV (Maize Chlorotic Mottle Virus).

An eIF(iso)4E protein variant able to confer resistance to at least one maize virus is thus able to confer resistance the disease resulting from an infection with said at least one maize virus.

The disease may for example be Maize Lethal Necrosis Disease (MLND), which is caused by the combination of (i) MCMV and (ii) SCMV, MDMV and WSMV.

By the expression “able to confer resistance to at least one maize virus”, it is herein meant that the expression of said eIF(iso)4E protein variant in a maize plant confers to said maize plant a resistance to said maize virus.

The ability of an eIF(iso)4E protein variant to confer resistance to a maize virus may be easily assessed by the skilled person, either in vitro or in vivo.

Resistance to a maize virus may for example be assessed in vivo by monitoring the development (or absence of development) of the maize disease resulting from an infection with said maize virus in maize plants genetically transformed or edited to express the eIF4E or eIFiso4E protein variant to be assessed. The maize virus is preferably inoculated to the plants, for example by mechanical inoculation. The results are preferably compared to wild-type maize plants placed in the same conditions. A decreased development of the maize viral disease or the absence of development of the maize viral disease in the maize plants expressing the eIF(iso)4E protein variant to be assessed by comparison to wild-type maize plants indicates that said eIF(iso)4E protein variant confers resistance to said maize virus.

Resistance to a maize virus may also be assessed by virus quantification, for example using Elisa or qPCR methods, which allow determining virus presence or absence and/or the quantity of virus in the maize plants expressing the eIF(iso)4E protein variant to be assessed by comparison to in wild-type maize plants. ELISA is able to measure the degree of virus accumulation, for example using antibodies specific for said maize virus. Anti-SCMV, anti-MDMV and anti-MCMV antibodies may for example be used.

Resistance to a maize viral disease may also be assessed in vitro, for example by yeast two hybrid system, wherein the possible binding of virus VPg to the eIF4E or eIFiso4E protein variant to be assessed is identified by yeast two hybrid assay. A protein variant which is not able to bind a maize virus VPg in this in vitro assay, while being expressed, is able to confer resistance to at said maize virus.

The VPg protein (Viral Protein genome-linked) is indeed a potyvirus protein, which binds to eIF(iso)4E proteins, said binding being required for viral infection. Thus, if the VPg protein is not able to bind to eIF(iso)4E proteins, the viral cycle is inhibited.

The eIF(iso)4E protein variant as defined above comprises at least one mutation, preferably at least two or at least three mutations by comparison to the corresponding native maize eIF(iso)4E protein.

The expression “the corresponding native maize eIF(iso)4E protein” means the native maize eIF4E1 protein for a eIF4E1 protein variant, the native maize eIF4E2 protein for a eIF4E2 protein variant, the native maize eIF(iso)4E1 protein for a eIF(iso)4E1 protein variant and the native maize eIF(iso)4E2 protein for a eIF(iso)4E2 protein variant.

By the term “mutation”, it is herein meant a substitution of one amino acid, a deletion of one amino acid or an addition of one amino acid.

Said at least one mutation, preferably said at least two or said at least three mutations, are preferably amino acid substitutions.

Said at least one mutation, preferably said at least two or said at least three mutations disrupt the interaction between (i) the eIF(iso)4E protein variant as defined above and (ii) at least one maize virus, such as a potyvirus, preferably, SCMV, MDMV or WSMV and/or a machlomovirus, such as MCMV. Said at least one mutation, preferably said at least two or said at least three mutations, particularly disrupt the interaction between (i) the eIF(iso)4E protein variant as defined above and (ii) the VPg (Viral Protein genome-linked) protein of said at least one maize virus as defined above.

The eIF(iso)4E protein variant as defined above is functional.

By “functional”, it is herein meant that, when present or expressed in a maize plant, the eIF(iso)4E protein variant has at least one biological function of the corresponding native eIF(iso)4E protein.

An example of biological function of the corresponding native eIF(iso)4E protein is protein synthesis.

Any suitable method may be used to assess if a eIF(iso)4E protein variant is functional, such as a complementation test, preferably in yeast.

In a yeast complementation test, the yeast is made deficient for the eIF(iso)4E protein. Said deficient yeast will grow only if the eIF(iso)4E protein variant is functional.

One, two or three consecutive amino acids may be mutated, preferably substituted, to disrupt said interaction.

When the protein variant comprises at least two mutations, at least two of said mutations may be located (i) in two consecutive amino acids or (ii) in two non-consecutive amino acids.

When the protein variant comprises at least three mutations, said at least three substitutions, may be located (i) in three consecutive amino acids, (ii) in two consecutive amino acids and one non-consecutive amino acid or (iii) in three non-consecutive amino acids.

The eIF4E protein variant as defined above may be eIF4E1 protein variant or eIF4E2 protein variant.

The eIFiso4E protein variant as defined above may be eIFiso4E1 protein variant or eIFiso4E2 protein variant.

Said native maize eIF4E protein is preferably protein eIF4E1 of sequence SEQ ID NO: 1 or protein eIF4E2 of sequence SEQ ID NO: 2.

Said native maize eIFiso4E protein is preferably protein eIFiso4E1 of sequence SEQ ID NO: 3 or protein eIFiso4E2 of sequence SEQ ID NO: 4.

The eIF(iso)4E protein variant as defined above may for example comprise at least one mutation, preferably at least two mutations or at least three mutations, for example at least four, at least five or at least six mutations selected from:
the group consisting of V96D, G97R, T7S, A19V, L331, N55D/K, P56S/T, A63D, A64D, S67A, D99G, S159K, Q164K, and G211L/D/S by reference to native maize protein of sequence SEQ ID NO: 1,
the group consisting of D101G, A65D, A66D, N57D/K, P58S/T, S69A, G98D, G99R, S161K, Q166K, G213L/D/S by reference to native maize protein of sequence SEQ ID NO: 2,
the group consisting of A60D, A61D, N94R, W108L, S157K and R161E/L by reference to native maize protein of sequence SEQ ID NO: 3, or
the group consisting of A54D, A55D, T88R, W102L, S151K and R155E/L by reference to native maize protein of sequence SEQ ID NO: 4.

Mutation “XNY” means a substitution of amino acid X at position N with amino acid Y.

Mutation “XNY/Z” means a substitution of amino acid X at position N with amino acid Y or Z.

The eIF4E protein variant as defined above may for example comprise at least one mutation selected from:
the group consisting of P56T, V96D, G97R, D99G and S159K, by reference to native maize protein of sequence SEQ ID NO: 1, or
the group consisting of A66D, G99R, D101G and S161K, by reference to native maize protein of sequence SEQ ID NO: 2.

Each of the mutations P56T, V96D, G97R, D99G, S159K, by reference to native maize protein of sequence SEQ ID NO: 1, particularly confers a resistance to SCMV. Mutation D99G by reference to native maize protein of sequence SEQ ID NO: 1 also confers a resistance to MDMV.

Each of the mutations G99R, D101G, S161K, by reference to native maize protein of sequence SEQ ID NO: 2, particularly confers a resistance to SCMV.

Each of the mutations A66D and D101G, by reference to native maize protein of sequence SEQ ID NO: 2, particularly confers a resistance to MDMV.

The eIF4E1 protein variant as defined above may for example comprise at least two mutations selected from the group consisting of:
N55K and P56S/T, which mutations particularly confer a resistance to SCMV,
N55D and P56T, which mutations particularly confer a resistance to SCMV,
A63D and A64D, which mutations particularly confer a resistance to SCMV and MDMV,
V96D and G97R, which mutations particularly confer a resistance to SCMV and MDMV,
S67A and S159K, which mutations particularly confer a resistance to SCMV,
D99G and Q164K, which mutations particularly confer a resistance to SCMV and MDMV, and
S159K and Q164K, which mutations particularly confer a resistance to SCMV, by reference to native maize protein of sequence SEQ ID NO: 1.

The eIF4E2 protein variant as defined above may for example comprise at least two mutations selected from the group consisting of:
A65D and A66D, which mutations particularly confer a resistance to SCMV and MDMV,
G98D and G99R, which mutations particularly confer a resistance to SCMV,
S69A and S161K, which mutations particularly confer a resistance to SCMV,
D101G and Q166K, which mutations particularly confer a resistance to SCMV and MDMV, and
S161K and Q166K, which mutations particularly confer a resistance to SCMV, by reference to native maize protein of sequence SEQ ID NO: 2.

The eIF4E1 protein variant as defined above may for example comprise at least three mutations selected from the group consisting of:
T7S, A19V and L331, which mutations particularly confer a resistance to SCMV,
N55K, P56T and G211S, which mutations particularly confer a resistance to SCMV,
N55K, P56T and S159K, which mutations particularly confer a resistance to SCMV,
A63D, A64D and G97R, which mutations particularly confer a resistance to SCMV and MDMV,
A63D, A64D and D99G, which mutations particularly confer a resistance to SCMV and MDMV,
A63D, A64D and S159K, which mutations particularly confer a resistance to SCMV and MDMV,
S67A, V96D and G97R, which mutations particularly confer a resistance to SCMV,
V96D, G97R and D99G, which mutations particularly confer a resistance to SCMV and MDMV, and
V96D, G97R and S159K, which mutations particularly confer a resistance to SCMV and MDMV,
   by reference to native maize protein of sequence SEQ ID NO: 1.

The eIF4E1 protein variant as defined above may for example comprise at least four mutations selected from the group consisting of:
A63D, A64D, G97R and S159K, which mutations particularly confer a resistance to SCMV and MDMV,
A63D, A64D, D99G and S159K, which mutations particularly confer a resistance to SCMV and MDMV,
A63D, A64D, S67A and S159K, which mutations particularly confer a resistance to SCMV and MDMV,
A63D, A64D, V96D and G97R, which mutations particularly confer a resistance to SCMV and MDMV,
N55K, P56T, A63D and A64D, which mutations particularly confer a resistance to SCMV and MDMV, and
N55D, P56S, A63D and A64D, which mutations particularly confer a resistance to SCMV and MDMV,
   by reference to native maize protein of sequence SEQ ID NO: 1

The eIF4E1 protein variant as defined above may for example comprise at least the following mutations:
A63D, A64D, V96D, G97R and S159K, which mutations particularly confer a resistance to SCMV and MDMV,
N55D, P56S, A63D, A64D, G97R and S159K, which mutations particularly confer a resistance to SCMV and MDMV,
N55D, P56S, A63D, A64D V96D, G97R and S159K, which mutations particularly confer a resistance to SCMV and MDMV,
N55D, P56S, A63D, A64D, V96D, G97R, S159K and G211S, which mutations particularly confer a resistance to SCMV and MDMV, or
N55K, P56T, A63D, A64D, V96D, G97R, S159K and G211D, which mutations particularly confer a resistance to SCMV and MDMV, by reference to native maize protein of sequence SEQ ID NO: 1.

The eIF4E2 protein variant as defined above may for example comprise at least three mutations selected from the group consisting of:
N57K, P58T and S161K, which mutations particularly confer a resistance to SCMV,
A65D, A66D and G99R, which mutations particularly confer a resistance to SCMV and MDMV,
A65D, A66D and S161K, which mutations particularly confer a resistance to SCMV and MDMV,
A65D, A66D and D101G, which mutations particularly confer a resistance to SCMV and MDMV,
G98D, G99R and D101G, which mutations particularly confer a resistance to SCMV and MDMV,
G98D, G99R and S161K, which mutations particularly confer a resistance to SCMV, and
S69A, G98D and G99R, which mutations particularly confer a resistance to SCMV, by reference to native maize protein of sequence SEQ ID NO: 2.

The eIF4E2 protein variant as defined above may for example comprise at least the following mutations:
A65D, A66D, D101G and S161K, which mutations particularly confer a resistance to SCMV and MDMV,
A65D, A66D, G99R and S161K, which mutations particularly confer a resistance to SCMV and MDMV,
A65D, A66D, S69A and S161K, which mutations particularly confer a resistance to SCMV and MDMV,
A65D, A66D, G98D and G99R, which mutations particularly confer a resistance to SCMV and MDMV,
N57K, P58T, A65D and A66D, which mutations particularly confer a resistance to SCMV and MDMV,
N57D, P58S, A65D and A66D, which mutations particularly confer a resistance to SCMV and MDMV,
D101G, S161K, Q166K and G213L, which mutations particularly confer a resistance to SCMV and MDMV,
N57D, P58S, G98D, G99R and S161K, which mutations particularly confer a resistance to SCMV,
A65D, A66D, G98D, G99R and S161K, which mutations particularly confer a resistance to SCMV and MDMV, or
N57K, P58T, G98D, G99R, S161K and G213D, which mutations particularly confer a resistance to SCMV,
   by reference to native maize protein of sequence SEQ ID NO: 2.

The eIF(iso)4E protein variant as defined above may for example comprise at least one mutation, preferably at least two mutations or at least three mutations, for example at least four, at least five or at least six mutations selected from:
the group consisting of A63D, A64D, V96D, G97R, D99G and G211L/D/S by reference to native maize protein of sequence SEQ ID NO: 1,
the group consisting of A65D, A66D, G98D, G99R, D101G and G213L/D/S by reference to native maize protein of sequence SEQ ID NO: 2,
the group consisting of A60D, A61D, N94R, W108L, S157K and R161E/L by reference to native maize protein of sequence SEQ ID NO: 3, or
the group consisting of A54D, A55D, T88R, W102L, S151K and R155E/L by reference to native maize protein of sequence SEQ ID NO: 4.

Mutations A63D, A64D, V96D, G97R and D99G by reference to sequence SEQ ID NO: 1 disrupt interaction between eIF4E1 and potyvirus, in particular both SCMV and MDMV. These mutations are homologous to mutations A65D, A66D, G98D, G99R by reference to SEQ ID NO: 2, which thus disrupt interaction between eIF4E2 and potyvirus, in particular both SCMV and MDMV.

Mutation G211L/D/S by reference to sequence SEQ ID NO: 1 disrupts interaction between eIF4E1 with MCMV. This mutation is homologous to mutation G213L/D/S by reference to sequence SEQ ID NO: 2, which thus disrupts interaction between eIF4E2 and MCMV.

Mutations A60D, A61D, N94R, W108L, S157K and R161E/L by reference to sequence SEQ ID NO: 3 disrupt interaction between eIFiso4E1 and a potyvirus, in particular MDMV. These mutations are homologous to mutations A54D, A55D, T88R, W102L, S151K, R155E/L by reference to sequence SEQ ID NO: 4, which thus disrupt interaction between eIFiso4E2 and potyvirus.

Mutations A63D and A64D by reference to sequence SEQ ID NO: 1 are homologous to mutations A65D and A66D by reference to sequence SEQ ID NO: 2, to mutations A60D, A61D by reference to sequence SEQ ID NO: 3 and to mutations A54D and A55D by reference to sequence SEQ ID NO: 4.

The eIF(iso)4E protein variant as defined above may for example comprise:
mutations (i) A63D and A64D, (ii) V96D and G97R, (iii) D99G and/or (iv) G211L/D/S, by reference to native maize protein of sequence SEQ ID NO: 1,
mutations (i) A65D and A66D, (ii) G98D and G99R, (iii) D101G and/or (iv) G213L/D/S, by reference to native maize protein of sequence SEQ ID NO: 2,
mutations (i) A60D and A61D, (ii) N94R, (iii) W108L and R161E/L and/or (iv) S157K, by reference to native maize protein of sequence SEQ ID NO: 3, or
mutations (i) A54D and A55D, (ii) T88R, (iii) W102L and R155E/L and/or (iv) S151K, by reference to native maize protein of sequence SEQ ID NO: 4.

A preferred eIF(iso)4E protein variant as defined above may for example comprise (i) at least one mutation disrupting interaction between said protein variant and at least one potyvirus and (ii) at least one mutation disrupting interaction between said protein variant and at least one machlomovirus.

A preferred eIF4E1 protein variant as defined above may for example comprise mutation G211L/D/S and at least two mutations selected from the group consisting of (i) A63D and A64D, (ii) V96D and G97R and (iii) D99G, by reference to native maize protein of sequence SEQ ID NO: 1.

A still preferred eIF4E1 protein variant as defined above may for example comprise mutations G211L/D/S, A63D and A64D and, optionally, at least one further mutation selected from the group consisting of (i) V96D and G97R and (ii) D99G, by reference to native maize protein of sequence SEQ ID NO: 1.

A preferred eIF4E2 protein variant as defined above may for example comprise mutation G213L/D/S and at least two mutations selected from the group consisting of (i) A65D and A66D, (ii) G98D and G99R and (iii) D101G, by reference to native maize protein of sequence SEQ ID NO: 2,

A still preferred eIF4E2 protein variant as defined above may for example comprise mutations G213L/D/S, A65D and A66D and, optionally, at least one further mutation selected from the group consisting of (i) G98D and G99R and (ii) D101G, by reference to native maize protein of sequence SEQ ID NO: 2.

A preferred eIFiso4E1 protein variant as defined above may for example comprise mutations A60D and A61D and at least one mutation selected from the group consisting of (i) N94R, (ii) W108L and R161E/L and (iii) S157K, by reference to native maize protein of sequence SEQ ID NO: 3, or

A preferred eIFiso4E2 protein variant as defined above may for example comprise mutations A54D and A55D and at least one mutation selected from the group consisting of (i) T88R, (ii) W102L and R155E/L and (iii) S151K, by reference to native maize protein of sequence SEQ ID NO: 4.

In a preferred embodiment, the eIF(iso)4E protein variant is an eIF4E1 protein variant comprising or consisting of sequence SEQ ID NO: 5, an eIF4E2 protein variant comprising or consisting of sequence SEQ ID NO: 6, an eIFiso4E1 protein variant comprising or consisting of sequence SEQ ID NO: 7 or an eIFiso4E2 protein variant comprising or consisting of sequence SEQ ID NO: 8.

### Genetically Modified Maize Cell

The present invention also relates to a genetically modified maize cell comprising at least one eIF(iso)4E protein variant able to confer resistance to at least one maize virus, in particular wherein said protein variant comprises at least one mutation by comparison to the corresponding native maize eIF(iso)4E.

The eIF(iso)4E protein variant is particularly as defined above.

As used herein, “maize cell” or “maize plant cell” particularly means a maize cell obtained from or found in a seed, suspension culture, embryo, meristematic region, callus tissue, leave, root, shoot, gametophyte, sporophyte, pollen or microspore. A maize cell also includes a protoplast, in particular obtained from a tissue as defined above, as well as a cell from our found in a plant cell tissue culture from which plants can be regenerated, or plant callus.

The expression “cell comprising at least one protein X” herein means a cell expressing said at least one protein X.

The genetically modified maize cell as defined above may for example comprise at least one exogenous nucleic acid encoding said eIF(iso)4E protein variant.

By “exogenous nucleic acid”, it is herein meant a nucleic acid, which is not naturally present in the maize cell. An exogenous nucleic acid can for example be introduced in a cell by genetic engineering.

The genetically modified maize cell as defined above preferably comprises at least one eIF(iso)4E protein variant and does not comprise any native eIF4E1 protein, nor any native eIF4E2 protein.

In one embodiment, the genetically modified maize cell as defined above preferably expresses at least one endogenous eIFiso4E protein. The maize cell as defined above for example expresses protein eIFiso4E1 and/or protein eIFiso4E2.

The genetically modified maize cell as defined above may for example comprise at least one eIF(iso)4E protein variant and at least one native eIFiso4E protein.

The genetically modified maize cell as defined above may for example comprise at least one eIF(iso)4E protein variant, a native eIFiso4E1 protein and a native eIFiso4E2 protein.

The genetically modified maize cell as defined above may comprise at least one eIF(iso)4E protein variant (preferably an eIF4E2 protein variant), a native eIFiso4E1 protein, a native eIFiso4E2 protein and a native eIF4E1 protein, and not comprise any eIF4E2 protein.

The genetically modified maize cell as defined above may comprise at least one eIF(iso)4E protein variant (preferably an eIF4E1 protein variant), a native eIFiso4E1 protein, a native eIFiso4E2 protein and a native eIF4E2 protein, and not comprise any eIF4E1 protein.

The genetically modified maize cell as defined above preferably comprises at least one eIF(iso)4E protein variant, a native eIFiso4E1 protein and a native eIFiso4E2 protein and does not comprise any native eIF4E1 protein, nor any native eIF4E2 protein.

In one embodiment the eIF(iso)4E protein variant results of endogenous gene edition.

Gene edition can be made by any suitable method well-known by the skilled person, such as using base editing, prime editing or targeted homologous recombination, in particular using specific nucleases, such as Meganuclease (such as I-Scel), Transcription Activator-Like Effector Nucleases (TALEN), Zinc Finger Nucleases (ZFNs) or Clustered regularly Interspaced Short Palindromic repeat associated nuclease Cas (CRISPR/Cas), such as CRISPR/Cas9 or CRISPR/Cas 12. Such methods also include physical or chemical mutagenesis.

In another embodiment, the eIF(iso)4E protein variant is exogenously expressed in the cell. The exogenous expression is, for example, driven through an expression vector, such as a vector comprising at least exogenous nucleic acid as defined above.

Advantageously, the genetically modified maize cell may further comprise at least one knocked out eif4e gene and/or at least mutated apb1 gene.

Knocking-out at least one eif4e gene may be advantageous to prevent expression of native eiF4E protein(s), in particular when the endogenous native eif4e1 gene and/or eif4e2 gene is/are still present in the cell. Knocking-out at least one eif4e gene may indeed further increase the resistance to virus conferred by the eiF(iso)4E protein variant. In one embodiment, the genetically modified maize cell as defined above comprises at least one eIF(iso)4E protein variant as defined above in the part “eIF(iso)4E protein variant” and at least one knocked out eif4e gene, in particular as described below.

In a specific embodiment, the genetically modified maize cell as defined above comprises at least one eIF(iso)4E protein variant as defined in the part “eIF(iso)4E protein variant” and a knocked out eif4e1 gene.

In another specific embodiment, the maize cell as defined above comprises at least one eIF(iso)4E protein variant as defined in the part “eIF(iso)4E protein variant” and one knocked out eif4e2 gene.

The eif4e gene, which is knocked out, is an endogenous eif4E gene.

By “endogenous gene”, it is herein meant a gene naturally present in the maize cell.

The eif4e gene to be knocked out may be eif4e1 gene of sequence SEQ ID NO: 9 or eif4e2 gene of sequence SEQ ID NO: 10.

The eif4e gene to be knocked out in the genetically modified maize cell for example comprises or consists of a sequence having at least 75%, at least 80%, at least 85%, at least 88%, at least 90%, at least 92%, at least 95%, at least 96%, at least 97%, at least 98% or at least 99% identity to SEQ ID NO: 9 or SEQ ID NO: 10.

In one embodiment, the eif4E gene, which is knocked out in the genetically modified maize cell comprises or consists of sequence SEQ ID NO: 9 or SEQ ID NO: 10.

The genetically modified maize cell as defined above may thus further comprise a knocked out eif4e1 gene and/or a knocked out eif4e2 gene.

By “knocked out gene”, it is herein meant a non-functional gene. A knocked-out gene cannot any more express a functional protein, meaning that full length protein initially encoded by the gene is not translated anymore, but that a non-functional protein can optionally be translated from said knocked out gene. The non-functional gene can also result in absence of transcription into RNA and/or translation into protein. By “non-functional protein”, it is herein meant a protein lacking at least one function of the functional protein, in particular of the native protein. Thus, a phenotype of decrease in protein level expression is not generated by a gene knock-out.

In the present application, the knock-out can be done by introducing at least one mutation, at least one other gene or sequence and/or at least a double stranded break in any part of the genomic sequence, including 5′ UTR, 3′UTR, introns, exons and promoter region of the eif4e gene.

The knock-out is preferably done by introducing at least one mutation, at least one other gene or sequence and/or at least a double stranded break in any part of the eif4e gene, more preferably inside introns and/or exons.

The gene may be knocked out by any suitable method well-known by the skilled person, such as using:
a nucleic acid comprising a mutation or a gene to be inserted in the eif4e gene by homologous recombination,
a site-specific nuclease for introducing a double-stranded break in the eif4e gene,
a zinc-finger nuclease comprising a DNA binding domain targeting the eif4e gene and a restriction endonuclease, thereby resulting in double stranded break in the eif4e gene,
TALEN (Transcription activator-like effector nuclease) comprising a DNA binding domain targeting the eif4e gene and a nuclease, thereby resulting in double stranded break in the eif4e gene, and/or
gene editing, for example using CRISPR/cas system, comprising a guide RNA complementary to a target sequence in the eif4e gene, said guide RNA being complexed with a Cas protein, thereby resulting in a double stranded break in the eif4e gene and optionally introducing a desired sequence.

When a double stranded break occurs in the eif4e gene, the cellular DNA repair mechanisms attempt to repair this double stranded break, usually through non-homologous end joining (NHEJ), which results in insertions-deletions (INDELs). INDELs may change the reading-frame and/or introduce non-sense mutation(s), thereby leading to a non-functional eif4e gene and thus to a disrupted or truncated protein.

In the present application, the term “gene editing” includes insertion, deletion or substitution of at least one base pair and leads to knock-out, for example by, change in the reading frame, specific amino acid change (including STOP codon creation) or a combination thereof. Gene editing involves breaking of DNA strands and DNA damages repair mechanism such as non-homologous end joining or homologous recombination.

Gene knockout is thus different from gene silencing.

Gene silencing is indeed the regulation of gene expression in a cell, to prevent the expression of a given gene. Gene silencing generally reduces the expression of a gene, but do not suppress totally (or entirely) its expression into a functional protein, contrary to gene knockout. Most popular gene silencing method is RNAi.

The expression “knocked-out gene” thus does not encompass a gene mutation resulting in a decrease in the level of expression of the protein encoded by said gene.

The genetically modified maize cell comprising a knocked out eif4e1 gene thus do not express any eIF4E1 protein.

The genetically modified maize cell comprising a knocked out eif4e2 gene thus do not express any eIF4E2 protein.

The maize cells as defined above may express a non-functional protein from the knocked-out eif4e gene.

By “non-functional protein encoded by the knocked-out eif4e gene”, it is herein meant a protein, which is not able to participate in protein synthesis.

Any suitable method may be used to assess if a protein encoded by a knocked-out eif4e gene is non-functional, such as a yeast complementation test or any known standard methods.

In a yeast complementation test, the yeast is made deficient for the eIF4E protein. Said deficient yeast will not grow if the protein encoded by a knocked-out eif4e gene is non-functional.

Said non-functional protein encoded by a knocked-out eif4e gene may for example consists of or comprises an amino acid sequence having at least 60%, at least 65%, at least 70%, at least 75%, at least 80%, at least 85%, at least 88%, at least 90%, at least 92%, at least 95%, at least 98% or at least 99% identity to SEQ ID NO: 13, SEQ ID NO: 14, SEQ ID NO: 15, SEQ ID NO: 16, SEQ ID NO: 17 or SEQ ID NO: 18.

Said non-functional protein encoded by a knocked-out eif4e gene may for example consists of or comprises an amino acid sequence selected from the group consisting of sequence SEQ ID NO: 13, SEQ ID NO: 14, SEQ ID NO: 15, SEQ ID NO: 16, SEQ ID NO: 17 and SEQ ID NO: 18.

The non-functional protein as defined above encoded by the knocked-out eif4e gene preferably has a length lower than 150 amino acids, preferably lower than 100 amino acids. In a preferred embodiment, the non-functional protein has a length lower than 100 amino acids and, consists or comprises an amino acid sequence having at least 60%, at least 65%, at least 70%, at least 75%, at least 80%, at least 85%, at least 88%, at least 90%, at least 92%, at least 95%, at least 98% or at least 99% identity to SEQ ID NO: 13, SEQ ID NO: 14, SEQ ID NO: 15,, SEQ ID NO: 17, or SEQ ID NO: 18.

Said non-functional protein may have a length lower than 100 amino acids and, consists of or comprises amino acid sequence SEQ ID NO: 13, SEQ ID NO: 14, SEQ ID NO: 15, SEQ ID NO: 17 or SEQ ID NO: 18.

The genetically modified maize cell may further comprise at least one apb1 mutated gene.

The apb1 gene is located on chromosome 3, more precisely at positions chr3: 134550012 to 134554530 in B73 genome version 4.

A reference sequence for the apb1 gene is for example sequence SEQ ID NO: 11.

The apb1 gene encodes protein APB1.

A reference sequence for native maize protein APB1 is sequence SEQ ID NO: 12, also referred to as Zm00001d041711_APB1 in B73 genome version 4.

apb1 gene is located between eif4e1 and eif4e2 loci. In some strains, regulator sequences in the promoter of the apb1 gene have been identified as responsible of resistance to the Sugarcane Mosaic Virus (SCMV), a Potyvirus ‘(see Leng et al. 2017).

The term “mutated gene” includes at least one mutation, such as an insertion, deletion and/or substitution, by comparison the wild-type gene, such as the apb1 gene of sequence SEQ ID NO: 11. This at least one mutation can occur in promoter, untranslated region, coding region (exon) and/or or non-coding region (intron) of the gene. Said mutated apb1 gene expression preferably increases the resistance to at least one maize virus conferred by the at least one eIF(iso)4E protein variant or leads to another virus infection resistance.

Preferably, the apb1 promoter comprises BoxII motif and three SP1 motifs.

In another embodiment, the genetically modified maize cell as defined above does not comprise any apb1 mutated gene.

The genetically modified maize cell as defined above may for example comprise:
at least one eiF4E1 protein variant, in particular expressed from a gene obtained by gene editing at the eif4E1 locus, a knocked out eif4e2 gene, a native eIFiso4E1 protein, a native eIFiso4E2 protein and, optionally, a mutated apb1 gene,
at least one eiF4E2 protein variant, in particular expressed from a gene obtained by gene editing at the eif4E2 locus, a knocked out eif4e1 gene, a native eIFiso4E1 protein, a native eIFiso4E2 protein and, optionally, a mutated apb1 gene,
at least one eiF4E1 protein variant, in particular obtained by transformation with a DNA sequence encoding the eIF4E1 protein variant integrated at a locus different from the eif4e1 locus, at least one knocked out eif4e gene, a native eIFiso4E1 protein, a native eIFiso4E2 protein and, optionally, a mutated apb1 gene,
at least one eiF4E1 protein variant, in particular obtained by transformation with a DNA sequence encoding the eIF4E1 protein variant integrated at a locus different from the eif4e1 locus, a knocked out eif4e1 gene, a knocked out eif4e2 gene, a native eIFiso4E1 protein, a native eIFiso4E2 protein and, optionally, a mutated apb1 gene,
at least one eiF4E2 protein variant, in particular obtained by transformation with a DNA sequence encoding the eIF4E2 protein variant integrated at a locus different from the eif4e2 locus, at least one knocked out eif4e gene, a native eIFiso4E1 protein, a native eIFiso4E2 protein and, optionally, a mutated apb1 gene,
at least one eiF4E2 protein variant, in particular obtained by transformation with a DNA sequence encoding the eIF4E2 protein variant integrated at a locus different from the eif4e2 locus, a knocked out eif4e1 gene, a knocked out eif4e2 gene, a native eIFiso4E1 protein, a native eIFiso4E2 protein and, optionally a mutated apb1 gene,
at least one eiF4E1 protein variant, in particular expressed from a gene obtained by gene editing at the eif4e1 locus, at least one eiF4E2 protein variant, in particular obtained by transformation with a DNA sequence encoding the eIF4E2 protein variant integrated at a locus different from the eif4e2 locus, a native eIFiso4E1 protein, a native eIFiso4E2 protein and, optionally, a mutated apb1 gene,
at least one eiF4E1 protein variant, in particular expressed from a gene obtained by gene editing at the eif4e1 locus, at least one eiF4E2 protein variant, in particular obtained by transformation with a DNA sequence encoding the eIF4E2 protein variant integrated at a locus different from the eif4e2 locus, a knocked out eif4e2 gene, a native eIFiso4E1 protein, a native eIFiso4E2 protein and, optionally, a mutated apb1 gene,
at least one eiF4E1 protein variant, in particular obtained by transformation with a DNA sequence encoding the eIF4E1 protein variant integrated at a locus different from the eif4e1 locus, at least one eiF4E2 protein variant, in particular expressed from a gene obtained by gene editing at the eif4e2 locus, a native eIFiso4E1 protein, a native eIFiso4E2 protein and, optionally, a mutated apb1 gene,
at least one eiF4E1 protein variant, in particular obtained by transformation with a DNA sequence encoding the eIF4E1 protein variant integrated at a locus different from the eif4e1 locus, at least one eiF4E2 protein variant, in particular expressed from a gene obtained by gene editing at the eif4e2 locus, a knocked out eif4e1 gene, a native eIFiso4E1 protein, a native eIFiso4E2 protein and, optionally, a mutated apb1 gene,
at least one eiF4E1 protein variant, in particular expressed from a gene obtained by gene editing at the eif4E1 locus, at least one eiF4E2 protein variant, in particular expressed from a gene obtained by gene editing at the eif4E2 locus, a native eIFiso4E1 protein, a native eIFiso4E2 protein and, optionally, a mutated apb1 gene, or
at least one eiF4E1 protein variant and at least one eiF4E2 protein variant, in particular both obtained by transformation with a DNA sequence encoding the eIF4E1 and eIF4E2 protein variants integrated at a locus different from the eif4e1 locus and from the eif4e2 locus, a knocked out eif4e1 gene, a knocked out eif4e2 gene, a native eIFiso4E1 protein, a native eIFiso4E2 protein and, optionally a mutated apb1 gene.

### Nucleic Acid and Vector

The present invention also relates to a nucleic acid encoding at least one eIF(iso)4E protein variant as defined above.

The nucleic acid as defined above may be provided in the form of a vector.

The present invention also relates to a vector.

The vector as defined above may be:
a) a vector suitable for integration of at least one nucleic acid encoding at least one eIF(iso)4E protein variant in the genome of a maize plant, or
b) a vector suitable for introducing at least one mutation in the sequence of an eIF(iso)4E gene, so that the obtained mutated eIF(iso)4E gene encodes an eIF(iso)4E protein variant as defined above.

Said vector suitable for introducing at least one mutation may for example be a vector suitable for targeted base editing or a vector suitable for prime editing.

In one embodiment, the vector is suitable for integration of at least one nucleic acid encoding at least one eIF(iso)4E protein variant in the genome of a maize plant, preferably at a targeted location in the genome, in particular by homologous recombination.

The vector suitable for integration comprises at least one nucleic acid encoding for eIF(iso)4E protein variant as defined above.

The nucleic acid encoding at least one eIF(iso)4E protein variant is under the control of a promoter, preferably a plant promoter, more preferably a promoter whose expression is compatible with the expression of the wild type eIF(iso)4E gene. In one specific embodiment, said promoter may comprise or consist of the 2000 nucleotides upstream of the ATG of an eIF(iso)4E gene.

The vector suitable for integration as defined above may further comprise a nucleic acid encoding for a Cas protein, for example a Cas9 or Cas12 protein, and at least one guide nucleotide, in particular designed to allow specifically cutting the genome at both 5′ and 3′ end of eIF(iso)4E gene to perform homologous recombination. The nucleic acid encoding a Cas protein, for example a Cas9 or Cas12 protein and the guide nucleotide(s) are preferably under the control of a constitutive promoter (for example ZmUbi). The vector suitable for integration as defined above may further comprise homologous sequences of both 5′ and 3′ end of the eIFiso4E gene to be replaced. The length of the homologous sequence is preferably of at least 250 nucleotides and at most 2000 nucleotides.

The vector suitable for targeted base editing as defined above may comprise a nucleic acid encoding for a Cas protein, for example a Cas9 or Cas12 protein used to a base editor module (preferably a deaminase) to convert a nucleotide to another type of nucleotide, such as A to T or A to G. The vector suitable for targeted base editing as defined above may further comprise at least one guide nucleic acid homologous to an upstream or downstream sequence of the nucleotide targeted for edition, in particular according to specific rules known from the skilled person. The nucleic acid encoding for a Cas protein for exampleCas9 or Cas12 protein and the guide nucleotide(s) are preferably under the control of a constitutive promoter (for example ZmUbi).

By “Cas protein”, it is herein meant an endonuclease of the CRISPR (Clustered Regularly Interspaced Short Palindromic Repeat)-Cas system.

The Cas protein of preferable a Cas9 or Cas12 protein.

By “Cas9 protein”, “CRISPR-associated protein 9” or “protein of type Cas9”, it is herein meant a dual RNA-guided DNA endonuclease.

By “Cas12 protein”, “CRISPR-associated protein 12” or “protein of type Cas12”, it is herein meant is a single RNA-guided endonuclease. Non-limiting examples of Cas9 or Cas12 protein as defined above comprise a nickase Cas9, a dead Cas9 or Cas12 (for example nCas9, nCas12, dCas9, dCas12), a PAM less Cas9 or Cas12.

The base editor module allows converting a nucleotide to another type of nucleotide according to the type of deaminase. The base editor may for example be a cytidine deaminase, adenosine deaminase or guanine deaminase.

The vector suitable for prime editing as defined above may comprise a nucleic acid encoding for a Cas protein, for example a Cas9 or Cas12 protein fused to a reverse transcriptase enzyme. The vector may further comprise a prime editing guide (pegRNA) capable of (i) identifying the target nucleotide sequence to be edited and (ii) encoding new genetic information to replace said target nucleotide sequence.

Said reverse transcriptase is preferably MMLV (Murine Leukemia Virus).

The vector suitable for prime editing as defined above may comprise a Cas9 nickase fused to the reverse transcriptase, preferably MMLV, under the control of the promoter of the maize actin gene and a prime editing guide.

The present invention also relates to a vector suitable for knocking out at least one maize eif4E gene, for example maize eIF4E1 gene and/or maize eIF4E2 gene.

The vector as defined above may be a plasmid.

The vector as defined above may comprise:
a) at least one CRISPR (Clustered Regularly Interspaced Short Palindromic Repeats)-Cas endonuclease expression cassette and/or
b) at least one gRNA expression cassette.

The vector as defined above preferably comprises:
a) at least one gRNA expression cassette, wherein said gRNA expression cassette comprises a nucleic acid encoding a gRNA under the control of a promoter and wherein said gRNA comprises a region complementary to a target region of the maize eif4E gene, and
b) optionally, at least one CRISPR-Cas endonuclease expression cassette.

Said at least one CRISPR-Cas endonuclease expression cassette and said at least one gRNA expression cassette may be provided in the same vector or in separate vectors.

The CRISPR-Cas endonuclease expression cassette comprises a nucleic acid encoding a Cas endonuclease under the control of a promoter.

The Cas endonuclease is an enzyme, which uses a gRNA as a guide to recognize and performs a double-stranded break at a specific position in a DNA sequence. The Cas endonuclease generally requires the presence of a Protospacer Adjacent Motif (PAM) sequence in the vicinity of the specific targeted position. The PAM sequence is preferably spaced from the targeted position by at most 20 nucleotides. The PAM sequence can differ depending on the Cas endonuclease.

When the Cas endonuclease requires a PAM sequence, the eif4E gene comprises a PAM sequence downstream the targeted region.

The Cas endonuclease may be selected from the group consisting of Cas9, Cas12a, Cas12b, C2c1 and C2c2.

The Cas endonuclease is preferably Cas9 (CRISPR-associated protein 9) endonuclease. Cas9 endonuclease may for example comprise or consist of sequence SEQ ID NO: 23.

The promoter of the CRISPR-Cas endonuclease expression cassette may be a constitutive promoter selected from the group consisting of ZmUbi promoter, the 35S promoter or the 19S promoter (Kay et al., 1987), the rice actin promoter (McElroy et al., 1990), the pCRV promoter (Depigny—This et al., 1992), the CsVMV promoter (Verdaguer et al., 1998) and the ubiquitin promoter from rice or sugarcane. The promoter of the CRISPR-Cas endonuclease expression cassette is preferably ZmUbi promoter.

The CRISPR-Cas endonuclease expression cassette preferably comprises a terminator, for example SbHSP.

The gRNA expression cassette comprises a nucleic acid encoding a gRNA under the control of a promoter.

The gRNA comprises:
a) a region complementary to a target region of the eif4E gene, preferably to a target region in an exon of the eif4E gene, and
b) a scaffold region, which allows binding to the CRISPR-Cas endonuclease encoded by the CRISPR-Cas endonuclease expression cassette.

The eif4E gene is particularly as defined above. The target region of the eif4E gene is preferably located in exon 1 or exon 2 of the eif4E gene. Exons 1 and 2 of eIF4E1 gene and those of eIF4E2 gene are particularly as defined above in the section “Maize eif4E genes and proteins”.

The promoter of the gRNA expression cassette may for example be selected from the group consisting of an RNA polymerase III promoter (for example TaU6 promoter, ZmU6 promoter or ZmU3), or an RNA polymerase II promoter, such as a constitutive promoter (for example ZmUbi or TaUbi). The promoter of the gRNA expression cassette is preferably ZmU6 promoter.

The gRNA produced by the gRNA expression cassette is thus able to recognize a target region of the eif4E gene, so that, when complexed to CRISPR-CAS endonuclease, a double-stranded break in introduced in the target region of the eif4E gene through the action of the CRISPR-CAS endonuclease.

The gRNA preferably comprises at least 15 nucleotides, preferably at least 17 nucleotides, more preferably at least 18 nucleotides and/or at most 25 nucleotides, preferably at most 24 nucleotides, more preferably at most 23 nucleotides.

The gRNA may for example comprise or consist of sequence SEQ ID NO: 19, SEQ ID NO: 20, SEQ ID NO: 21 or SEQ ID NO: 22.

### Genetically Modified or Edited Maize Plant or Seed Resistant to at Least One Maize Virus

The present invention also relates to a genetically modified or edited maize plant resistant to at least one maize virus or a genetically modified or edited maize seed resistant to at least one maize virus.

Said maize virus is particularly as defined above.

Said genetically modified or edited maize plant or genetically modified or edited maize seed is thus resistant to the disease caused by said at least one maize virus.

Said disease is for example as defined above.

The genetically modified or edited maize plant or genetically modified or edited maize seed as defined above may for example be resistant to MNLD.

By “genetically modified” or “GM”, it is herein meant that the integration of the modification is done at any location in the genome. In a GM plant or GM seed, the wild type gene is thus present.

By “genetically editing” or “GE”, it is herein meant that the modification is done at the locus of origin. In a GE plant or GE seed, the wild type gene is thus no more present.

Said GM or GE plant resistant to at least one maize virus or said GM or GE seed resistant to at least one maize virus expresses at least one eIF(iso)4E protein variant as defined above. In other word, the cells of said plant or seed express at least one eIF(iso)4E protein variant as defined above.

The plant or seed as defined above comprises genetically modified maize cells, in particular as defined in the part “Genetically modified maize cell”.

The plant or seed as defined above preferably comprises genetically modified maize cells, wherein said cells comprise at least one eIF(iso)4E protein variant and do not comprise any native eIF4E1 protein, nor any native eIF4E2 protein.

The cells of the plant or seed as defined above preferably comprise at least one nucleic acid as defined above encoding at least one eIF(iso)4E protein variant as defined above.

Said nucleic acid is preferably inserted in the genome of the cells of said plant or seed.

Said nucleic acid is preferably replacing (in particular by allele exchange) the corresponding endogenous eIF(iso)4E gene.

The maize plant or seed as defined above is preferably an agronomic plant or seed.

By the expression “agronomic plant or seed”, it is herein meant a plant or seed suitable for production on a large scale, in particular for human and animal food or for industrial purposes.

In one embodiment, the plant or seed resistant to at least one maize virus as defined above is not a null mutant of any or all eIF(iso)4E genes, since said plant or seed expresses at least one eIF(iso)4E protein variant.

### Method for Obtaining a Maize Plant Resistant to at Least One Maize Virus

The present invention particularly relates to a method for obtaining a maize plant resistant to at least one maize virus, in particular as defined above, wherein said method comprises:
(i) introducing at least one nucleic acid encoding at least one eIF(iso)4E protein variant in the genome of a maize cell or a maize tissue or (ii) introducing at least one mutation in the endogenous eIF(iso)4E gene of a maize cell or a maize tissue, so that the obtained mutated eIF(iso)4E gene encodes at least one eIF(iso)4E protein variant as defined above, and
regenerating a maize plant from the obtained maize cell or maize tissue.

Methods for introducing at least one nucleic acid in the genome of a maize cell or a maize tissue are well known by the skilled person.

Introducing at least one nucleic acid encoding at least one eIF(iso)4E protein variant in the genome of a maize cell or a maize tissue may for example comprise transforming a maize cell or a maize tissue with a vector comprising at least one nucleic acid encoding at least one eIF(iso)4E protein variant.

Methods for introducing at least one mutation in an endogenous plant gene are well known by the skilled person. Such methods for example include base editing, prime editing, or targeted homologous recombination, in particular using specific nucleases, such as Meganuclease (such as I-Scel), Transcription Activator-Like Effector Nucleases (TALEN), Zinc Finger Nucleases (ZFNs) or Clustered regularly Interspaced Short Palindromic repeat associated nuclease Cas (CRISPR/Cas), such as CRISPR/Cas9 or CRISPR/Cas 12. Such methods also include physical or chemical mutagenesis.

Introducing at least one mutation in an endogenous maize gene encoding an eIF(iso)4E protein as defined above may comprise transforming a maize cell or a maize tissue with a vector suitable for introducing at least one mutation in the endogenous eIF(iso)4E gene, so that the obtained mutated eIF(iso)4E gene encodes at least one eIF(iso)4E protein variant as defined above.

The present invention thus particularly relates to a method for obtaining a maize plant resistant to at least one maize virus, in particular as defined above, wherein said method comprises transforming a maize cell or a maize tissue with (i) a vector comprising at least one nucleic acid encoding at least one eIF(iso)4E protein variant or (ii) a vector suitable for introducing at least one mutation in the endogenous eIF(iso)4E gene, so that the obtained mutated eIF(iso)4E gene encodes at least one eIF(iso)4E protein variant as defined above.

The vector may be a vector as defined above, such as a vector suitable for inserting at least one nucleic acid encoding at least one eIF(iso)4E protein variant as defined above in the genome of maize, a vector suitable for targeted base editing, or a vector suitable for prime editing.

In one preferred embodiment, the nucleic acid encoding an eIF(iso)4E protein variant is introduced at a specific genome location in the maize cell or tissue.

The method as defined above preferably allows replacing the endogenous eIF(iso)4E gene with at least one nucleic acid coding for the eIF(iso)4E protein variant, in particular by homologous recombination at the same genome location.

The nucleic acid encoding an eIF(iso)4E protein variant is preferably as defined above. Said nucleic acid may be provided in the form of a vector as defined above.

In another preferred embodiment, the method as defined above allows mutating at least one nucleotide of the endogenous eIF(iso)4E gene, for example by gene editing or prime editing, to obtain a mutated eIF(iso)4E gene encoding an eIF(iso)4E protein variant as defined above.

Said maize virus is particularly as defined above.

Resistance to said at least one maize virus confers resistance to the disease caused by said maize virus.

Said disease is particularly as defined above.

Any method well known by the skilled person for introducing a nucleic acid of interest in the genome of a plant may be used.

Any method well known by the skilled person for replacing a nucleic acid by another nucleic of interest in a plant (such as an allele exchange) may be used.

Any method well known by the skilled person for introducing a mutation of interest in the genome of a plant may be used.

The method for obtaining a maize plant resistant to at least one maize virus as defined above may for example comprise:
a) transforming a maize cell or maize tissue with at least one vector as defined above in the section “Vector”, in particular comprising a nucleic acid encoding an eIF(iso)4E protein variant as defined above or with a vector suitable for introducing at least one mutation in the endogenous eIF(iso)4E gene, so that the obtained mutated eIF(iso)4E gene encodes at least one eIF(iso)4E protein variant as defined above, to obtain a transformed maize cell or a transformed maize tissue,
b) regenerating a maize plant from the transformed maize cell or transformed maize tissue, and
c) optionally, assessing the expression of said eIF(iso)4E protein variant by the regenerated maize plant.

### Step a)

Step a) comprises transforming a maize cell or a maize tissue with a vector.

The maize cell may be a protoplast.

The maize tissue may be an apical meristem, cotyledon, embryo (preferably an immature embryo), pollen and/or microspores.

The maize tissue is preferably an immature embryo of 10 days after fertilization.

The maize cell or maize tissue used as starting material comprise at least one knocked out eif4e gene and/or a mutated apb1 gene, in particular as defined above.

Any technique suitable for plant cell or plant tissue transformation may be used, such as biolistic particle delivery, PEG transformation, electroporation or agrobacterium transgene delivery.

For agrobacterium transgene delivery, the vector is first transferred into Agrobacterium, to obtain a transformed Agrobacterium and the maize cell or maize tissue is then transformed with said transformed Agrobacterium. The Agrobacterium is preferably Agrobacterium tumefaciens.

Transformation with said transformed Agrobacterium for example comprises co-culturing the maize cell or maize tissue with Agrobacterium, for example for at least 5 minutes.

Alternatively to transformation, introduction of the vector can be done by using haploid inducer line or associated technologies.

When the vector comprises a nucleic acid encoding an eIF(iso)4E protein variant, the transformed maize cell or transformed maize tissue obtained in step a) thus expresses said eIF(iso)4E protein variant.

If needed, the maize cell or maize tissue of step a) or the transformed maize cell or transformed maize tissue of step a) may be knocked out for at least one eIF4E gene, in particular so that transformed maize cell or transformed maize tissue used in step b) does not express any native eIF4E1 protein, nor any native eIF4E2 protein.

The method as defined above may thus optionally comprise before step a), during step a) or after step a) a step of knocking out at least one eif4e gene in a maize cell or maize tissue, to obtain a knocked out maize cell or a knocked out maize tissue.

The method as defined above may thus comprises:
before step a), a step of knocking out at least one eIF4E gene in a maize cell or maize tissue, to obtain a knocked out maize cell or a knocked out maize tissue, which is the maize cell or maize tissue used in step a), or
after step a) and before step b), a step of knocking out at least one eif4e gene in a transformed maize cell or transformed maize tissue, to obtain a knocked out transformed maize cell or a knocked out transformed maize tissue, which is the transformed maize cell or transformed maize tissue used in step b).

Alternatively, step a) comprises (i) transforming a maize cell or maize tissue with at least one vector as defined above in the section “Vector”, in particular comprising a nucleic acid encoding an eIF(iso)4E protein variant as defined above or with a vector suitable for introducing at least one mutation in the endogenous eIF(iso)4E gene, so that the obtained mutated eIF(iso)4E gene encodes at least one eIF(iso)4E protein variant as defined above and (ii) knocking out at least one eif4E gene in said maize cell or maize tissue, to obtain a transformed maize cell or a transformed maize tissue.

The eif4E gene is an endogenous eif4E gene, in particular as defined above.

The eif4E gene may be knocked out by any suitable method well-known by the skilled person as defined above, such:
homologous recombination with a nucleic acid comprising a mutation or a gene to be inserted in the eIF4E gene,
double-stranded break introduced in the eif4E gene by a site-specific nuclease, a zinc-finger nuclease comprising a DNA binding domain targeting the eif4E gene and a restriction endonuclease, a TALEN (Transcription activator-like effector nuclease) comprising a DNA binding domain targeting the eif4E gene and a nuclease or by gene editing.

The eIF4E gene is preferably knocked out by gene editing, more preferably using CRISPR/Cas system.

The step of knocking out at least one eif4E gene preferably comprises introducing into said maize cell (or transformed maize cell) or maize tissue (or transformed maize tissue) preferably at least one vector suitable for knocking out at least one maize eif4E gene.

Any method for introducing a nucleic acid or a vector in a cell or a tissue may be used, such as transformation or using a haploid inducer line.

Transformation and the use of a haploid inducer line are as defined above.

Said vector suitable for knocking out at least one maize eif4E gene is for example as defined above.

When more than one vector is used in step a), the maize cell or maize tissue is preferably transformed with the different vectors at the same time.

When using a vector suitable for knocking out at least one maize eif4E gene as defined above, step b) particularly results in:
the expression of the Cas endonuclease(s) from the CRISPR-Cas endonuclease expression cassette(s) and the production of gRNA(s) from the gRNA expression cassette(s), thereby producing a double strand break in the eif4E gene in the plant genome, and
double-strand break repair, in particular by non-homologous end joining (NHEJ), leading to insertions and/or deletions in the targeted eif4E gene.

When the maize cell or maize tissue is transformed with at least one vector comprising a nucleic acid encoding an eIF4E1 protein variant suitable for homologous recombination at the eif4e1 locus (thereby replacing the endogenous eif4e1 gene) or with a vector suitable for introducing at least one mutation in the endogenous eif4e1 gene as defined above, the method as defined above preferably comprises knocking out the eif4e2 gene or uses (as starting material) a maize cell or maize tissue knocked out for the eif4e2 gene.

When the maize cell or maize tissue is transformed with at least one vector comprising a nucleic acid encoding an eIF4E2 protein variant suitable for homologous recombination at the eif4e2 locus (thereby replacing the endogenous eif4e2 gene) or with a vector suitable for introducing at least one mutation in the endogenous eif4e2 gene as defined above, the method as defined above preferably comprises knocking out the eif4e1 gene or uses (as starting material) a maize cell or maize tissue knocked out for the eif4e1 gene.

When the maize cell or maize tissue is transformed with (i) at least one vector comprising a nucleic acid encoding an eIF4E1 protein variant suitable for homologous recombination at the eif4e1 locus (thereby replacing the endogenous eif4e1 gene) or with a vector suitable for introducing at least one mutation in the endogenous eif4e1 gene and (ii) at least one vector comprising a nucleic acid encoding an eIF4E2 protein variant suitable for homologous recombination at the eif4e2 locus (thereby replacing the endogenous eif4e2 gene) or with a vector suitable for introducing at least one mutation in the endogenous eif4e2 gene, the method as defined above does not comprise knocking out the eif4e1 gene, nor the eif4e2 gene.

When the maize cell or maize tissue is transformed with at least one vector comprising (i) a nucleic acid encoding an eIF4E1 protein variant suitable for homologous recombination at the eif4e1 locus (thereby replacing the endogenous eif4e1 gene) and (ii) a nucleic acid encoding an eIF4E2 protein variant suitable for homologous recombination at the eif4e2 locus (thereby replacing the endogenous eif4e2 gene) or with a vector suitable for introducing at least one mutation in the endogenous eif4e1 gene and at least one mutation in the endogenous eif4e2 gene, the method as defined above does not comprise knocking out the eif4e1 gene, nor the eif4e2 gene.

### Step b)

Step b) comprises regeneration of a maize plant.

Regeneration of a plant from a plant cell or plant tissue is well known by the skilled person.

In particular, the maize cell or maize tissue may be placed in a culture medium suitable for maize growth.

The regeneration of a maize plant from a maize cell or a maize tissue may comprise:
growing said maize cell or maize tissue to obtain a callus, and
regeneration of shoots from the callus, in particular by somatic embryogenesis.

The growth of the maize cell into a callus and the regeneration of shoots, in particular by somatic embryogenesis, are carried out in any suitable culture medium comprising plant growth regulators. Induction of somatic embryogenesis can be activated by genes encoding transcription factors, such as BABY BOOM (bbm) (Horstman et al., 2017 October; 175 (2): 848-857. doi: 10.1104/pp. 17.00232. Epub 2017 Aug. 22).

The regeneration of a maize plant from a maize tissue may comprise regeneration of shoots.

The regeneration of shoots from the maize tissue may be carried out in any suitable culture medium comprising plant growth regulators.

### Step c)

The method may further comprise a step c) of assessing the expression of said eIF(iso)4E protein variant by the regenerated maize plant.

Assessing the expression of said eIF(iso)4E protein variant by the regenerated maize plant may be performed by:
assessing the resistance of the regenerated maize plant to at least one maize virus, for example as disclosed above,
detecting the expression of the eIF(iso)4E protein variant by the cells of the regenerated maize plant, in particular according to any method well-known by the skilled person, such as western-blot or an immunoassay, and/or
assessing if the cells of the regenerated maize plant comprise a nucleic acid encoding said eIF(iso)4E protein variant, in particular according to any method well-known by the skilled person, such as sequencing or PCR.

Optionally, step c) may further comprise assessing that at least one eif4e gene is knocked out in the regenerated maize plant, for example by assessing if the cells of the regenerated maize plant comprise a nucleic acid encoding non-functional eIF4E protein, in particular according to any method well-known by the skilled person, such as sequencing or PCR, or by detection the expression of a non-functional eIF4E protein by the cells of the regenerated maize plant, in particular according to any method well-known by the skilled person, such as western-blot or an immunoassay.

The regenerated maize plant expresses said eIF(iso)4E protein variant if:
the regenerated maize plant is resistant to at least one maize virus,
the cells of the regenerated maize plant express said eIF(iso)4E protein variant, and/or
the cells of the regenerated maize plant comprise a nucleic acid encoding said eIF(iso)4E protein variant.

The regenerated maize plant is knocked out for at least one eIF4E gene if:
the cells of the regenerated maize plant express an eIF4E non-functional protein, and/or
the cells of the regenerated maize plant comprise a nucleic acid encoding a eIF4E non-functional protein.
   Use of at Least One eIF(Iso)4E Protein Variant for Preventing at Least One Viral Disease in Maize

The present invention also relates to the use of at least one eIF(iso)4E protein variant for preventing at least one viral disease in maize.

Said viral disease is particularly as defined above.

Said viral disease is particularly caused by a maize virus as defined above.

Said viral disease is preferably caused by a potyvirus and/or a machlomovirus.

The invention particularly relates to the use as defined above for preventing MNLD, in particular MNLD caused by MCMV and at least one potyvirus, such as SCMV or MDMV.

The eIF(iso)4E protein variant is as defined above.

The present invention particularly relates to the use as defined above, wherein said at least one eIF(iso)4E protein variant is expressed in the maize, in particular in the maize plant or maize seed, in other words in the cells of said maize plant or maize seed.

The present invention also relates to the use of at least one eIF(iso)4E protein variant for conferring maize resistance to at least one maize virus, preferably at least one potyvirus (such as SCMV or MDMV) and/or at least one machlomovirus (such as MCMV), in particular wherein said at least one eIF(iso)4E protein variant is expressed in the maize, in particular in the maize plant or maize seed, in other words in the cells of said maize plant or maize seed.

The eIF(iso)4E protein variant is as defined above.

### Method for Identifying a Maize Plant Resistant to at Least One Maize Virus

The present invention also related to a method for identifying a maize plant resistant to at least one maize virus, wherein said method comprises detecting the expression of an eIF(iso)4E protein variant as defined above and/or the presence of a nucleic acid encoding an eIF(iso)4E protein variant as defined above and optionally, the expression of a non-functional eIF4E protein and/or the presence of a nucleic acid encoding a non-functional eIF4E protein and/or the presence of a nucleic acid that does not enable any eif4e gene expression in the cells of said maize plant.

Said maize virus is as defined above.

Detecting the expression of an eIF(iso)4E protein variant or a non-functional eIF4E protein may be performed as defined above.

The nucleic acid encoding an eIF(iso)4E protein variant or a non-functional eIF4E protein may be a DNA or a RNA.

Detecting the presence of a nucleic acid encoding an eIF(iso)4E protein variant or the presence of a nucleic acid encoding a non-functional eIF4E protein or the presence of a nucleic acid that does not enable any eif4e gene expression may be performed by any method well known by the skilled person, such as using at least one pair of PCR primers.

Said pair of PCR primers may be able to amplify a region comprising a portion of the endogenous plant genome and at least a portion of the nucleic acid encoding the eIF(iso)4E protein variant or encoding a non-functional eIF4E protein or nucleic acid that does not enable any eif4e gene expression.

Said pair of PCR primers is preferably able to detect the presence of the eIF(iso)4E protein variant, but not of the corresponding wild type eIF(iso)4E protein.

The expression of an eIF(iso)4E protein variant as defined above and/or the presence of a nucleic acid encoding an eIF(iso)4E protein variant as defined above in the cells of a maize plant indicates that said maize plant is resistant to at least one maize virus.

The invention will be further illustrated in the following figures and examples.

### BRIEF DESCRIPTION OF THE SEQUENCES

SEQ ID NO: 1 corresponds the sequence of a native maize eIF4E1 protein.

SEQ ID NO: 2 corresponds the sequence of a native maize eIF4E2 protein.

SEQ ID NO: 3 corresponds the sequence of a native maize eIFiso4E1 protein.

SEQ ID NO: 4 corresponds the sequence of a native maize eIFiso4E2 protein.

SEQ ID NO: 5 corresponds the sequence of a maize eIF4E1 protein variant comprising mutations A63D, A64D, V96D, G97R, D99G and G211L.

SEQ ID NO: 6 corresponds the sequence of a maize eIF4E2 protein variant comprising mutations A65D, A66D, G98D, G99R, D101G and G213L.

SEQ ID NO: 7 corresponds the sequence of a maize eIFiso4E1 protein variant comprising mutations A60D, A61D, N94R, W108L, S157K and R161E.

SEQ ID NO: 8 corresponds the sequence of a maize eIFiso4E2 protein variant comprising mutations A54D, A55D, T88R, W102L, S151K and R155E.

SEQ ID NO: 9 correspond to genomic sequence of maize eif4e1 gene.

SEQ ID NO: 10 corresponds to genomic sequence maize of eif4e2 gene.

SEQ ID NO: 11 corresponds to genomic sequence of maize apb1 gene.

SEQ ID NO: 12 corresponds to the sequence of a native maize APB1 protein.

SEQ ID NO: 13 corresponds to the sequence of the eIF4E1 protein allele 9_16.

SEQ ID NO: 14 corresponds to the sequence of a non-functional eIF4E1 protein obtained after knocking out.

SEQ ID NO: 15 corresponds to the sequence of another non-functional eIF4E1 protein obtained after knocking out.

SEQ ID NO: 16 corresponds to the sequence of a non-functional eIF4E2 protein obtained after knocking out.

SEQ ID NO: 17 corresponds the sequence of another non-functional eIF4E2 protein obtained after knocking out.

SEQ ID NO: 18 corresponds to the sequence of another non-functional eIF4E2 protein obtained after knocking out.

SEQ ID NO: 19 corresponds to the sequence of gRNA14 (ZmeIF4E_a_SpCas9_gRNA14) targeting a region in exon 1 of eIF4E1.

SEQ ID NO: 20 corresponds to the sequence of gRNA47 (ZmeIF4E_b_SpCas9_gRNA47) targeting a region in exon 1 of eIF4E2.

SEQ ID NO: 21 corresponds to the sequence of gRNA ZmeIF4E_a324&b330_SpCas9_gRNA targeting a region in exon 2 of eIF4E1 and eIF4E2.

SEQ ID NO: 22 corresponds to the sequence of gRNA ZmeIF4E_a367&b373_SpCas9_gRNA targeting a region in exon 2 of eIF4E1 and eIF4E2.

SEQ ID NO: 23 corresponds to the sequence of the V40 NLS-SpCas9_Nucleoplasmin NLS, a Cas9 (CRISPR-associated protein 9) endonuclease.

SEQ ID NO: 24 corresponds to U6 promoter sequence from maize.

SEQ ID NO: 25 corresponds to Ubiquitin promoter and 5′UTR sequence from maize. SEQ ID NO: 26 to 47 correspond to the epegRNAs designs of Table 5.

### DESCRIPTION OF THE FIGURES

FIG. 1: Growth of yeast cells co-transformed with (i) pGBKT7-SCMV-VPg and (ii) pGADT7-eIF4E1, pGADT7-eIF4E2, pGADT7-eIFiso4E1 or pGADT7-eIFiso4E2. Used of the prey vector pGADT7 (activation domain (AD) with eIF(iso)4E protein) and bait vector pGBKT7 (binding domain (BD) with VPg protein) on selective media.

FIG. 2: Growth of yeast cells co-transformed with (i) pGBKT7-MDMV-VPg and (ii) pGADT7-eIF4E1, pGADT7-eIF4E2, pGADT7-eIFiso4E1 or pGADT7-eIFiso4E2. Used of the prey vector pGADT7 (activation domain (AD) with eIF(iso)4E protein) and bait vector pGBKT7 (binding domain (BD) with VPg protein) on selective media.

FIG. 3: Growth of yeast cells co-transformed with (i) pGBKT7-SCMV-VPg and (ii) pGADT7-eIF4E1, pGADT7-eIF4E1_varA63D and PGADT7-eIF4E1_varA63D/A64D on selective media (SD-LW: selective media without Leucine & Tryptophane; SD-LWH: selective media without Leucine, Tryptophane & Histidine; SD-LWHA: selective media without Leucine, Tryptophane, Histidine & Adenine.

FIG. 4: MDMV infection rate in plants J1A, plants expressing native eiF4E1 protein (Zm00001d041682_eIF4E1_Native) and plants expressing eiF4E1 protein variants (Zm00001d041682_eIF4E1_Var1, Zm00001d041682_eIF4E1_Var4 and Zm00001d041682_eIF4E1_Var5).

### EXAMPLES

### Example 1: Identification of eIF(Iso)4E Genes in Maize and Selection of Variants Candidate for Resistance to Viruses

Identification of maize eIF(iso)4E genes has been done by blast method using eIF(iso)4E protein sequences of other species as bait. Two eIF4E and two eIFiso4E genes in maize genome of B73 on chromosome 3 named eIF4E1 and eIF4E2 were identified:
Zm00001d041682 as eIF4E1
Zm00001d041973 as eIF4E2
Zm00001d032775 as eIFiso4E2
Zm00001d014065 as eIFiso4E1.

### Example 2: Yeast Two-Hybrid results to identify VPg-eIF(iso)4E interaction

For the Y2H (Yeast Two-Hybrid) assay, the activation domain (AD) vector pGADT7 and the GAL4 DNA-binding domain (BD) vector pGBKT7 were used (Matchmaker Gold Yeast Two-Hybrid System (Clontech, Mountain View, CA, USA). The prey vector pGADT7 (activation domain (AD) with eIF(iso)4E protein) and bait vector pGBKT7 (binding domain (BD) with VPg protein) harboring the genes to be tested were co-transformed pairwise into the yeast (Saccharomyces cerevisiae) strains Y187 & Y2HGold.

Y2H based on GAL4 was done to identify the interaction between SCMV-/MDMV-VPg with the two ZmeIF4E and the two ZmeIFiso4E homologs. The bait vectors pGBKT7-SCMV-VPg, pGBKT7-SrMV-VPg and pGBKT7-SCSMV-VPg were individually co-transformed with the prey vectors pGADT7-eIF4E1, pGADT7-eIF4E2, pGADT7-eIFiso4E1, pGADT7-eIFiso4E2 into the Saccharomyces cerevisiae strains Y187 & Y2HGold.

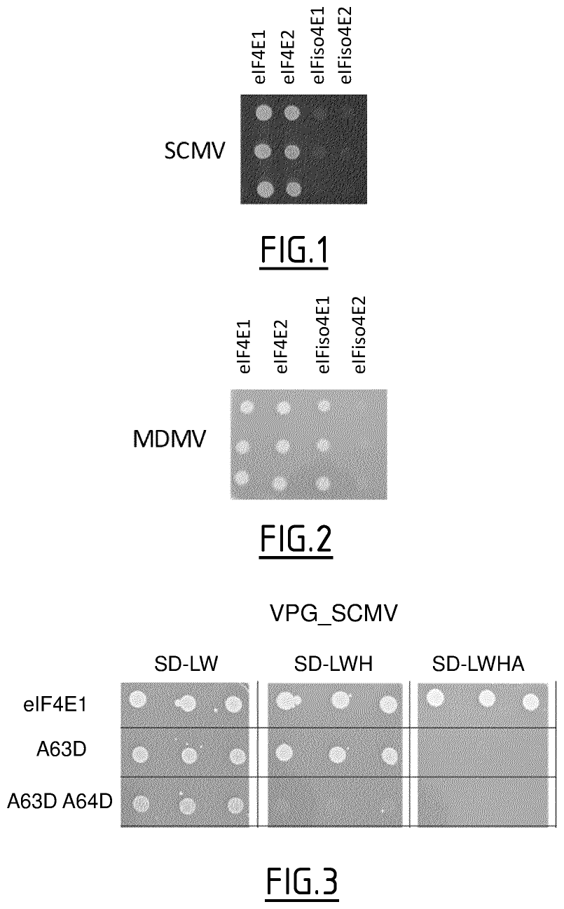

As a result, the yeast cells co-transformed with pGBKT7-SCMV-VPg and pGADT7-eIF4E1 or pGADT7-eIF4E2 grow well which indicate an interaction between SCMV-VPg and eIF4E proteins, whereas pGADT7-eIFiso4E1 and pGADT7-eIFiso4E2 no yeast cells growing were, indicating no interaction between SCMV-VPg and eIFiso4E proteins (see FIG. 1).

For MDMV, pGBKT7-MDMV-VPg and pGADT7-eIF4E1 or pGADT7-eIF4E2 grow well which indicate an interaction between MDMV and eif4E genes, the same result was obtained for pGADT7-eIFiso4E1. Whereas with pGADT7-eIFiso4E2, no yeast cells growing were, indicating no interaction between MDMV-VPg and eIFiso4E2 genes (see FIG. 2).

### Example 3: Identification by Yeast Two Hybrid of eIF(Iso)4E Mutations Disrupting VPg-eIF(Iso)4E Interaction

Following previous results, amino-acid modifications were introduced in eIF(iso)4E genes from maize to determine mutation impact on VPg-eIF(iso)4E genes interaction. The objective was to identify ones which disrupt the interaction between virus and host factor. As an example, A63D and A63D/A64D modify VPg-eIF4E1 interaction (see FIG. 3).

As a result, tables 3 and 4 below summarize all mutations tested (single or in combination) and tables 1 and 2 below indicate the organism source of the tested mutations, when relevant.

These set of data allowed us to identify mutations of interest that disrupt interaction with MDMV and/or SCMV.

TABLE 1
eIF4E1 mutations

| | N-Term | | | Region I | | | | | | |
|---|---|---|---|---|---|---|---|---|---|---|
| Mutation | T7S | A19V | L33I | N55D | N55K | P56S | P56T | A63D | A64D | S67A |
| Origine | Z. | Z. | Z. | C. | C. | C. | S | P. | P. | P. |
| | maize | maize | maize | annuum | annuum | annuum | lycopersicum | sativum, | sativum, | sativum |
| | | | | | | | | C. | C. | |
| | | | | | | | | annuum, | annuum, | |
| | | | | | | | | A | A | |
| | | | | | | | | thaliana | thaliana, | |
| | | | | | | | | | S | |
| | | | | | | | | | lycopersicum | |

| | Region II | | | Region III | | | | |
|---|---|---|---|---|---|---|---|---|
| Mutation | V96D | G97R | D99G | S159K | Q164K | G211D | G211L | G211S |
| Origine | H. | P. | S | P. | H. | H. | C | H. |
| | vulgare | sativum, | lycopersicum | sativum, | vulgare | vulgare | Melo | vulgare, |
| | | C. | | A. | | | | S |
| | | annuum, | | thaliana | | | | lycopersicum |
| | | A | | | | | | |
| | | thaliana | | | | | | |

TABLE 2
eIF4E2 mutations

| | Region I | | | | | | | Region II |
|---|---|---|---|---|---|---|---|---|
| Mutation | N57D | N57K | P58S | P58T | A65D | A66D | S69A | G98D |
| Origine | C. | S | C. | S | P. | P. | P. | H. |
| | annuum | lycopersicum | annuum | lycopersicum | sativum, | sativum, | sativum | vulgare |
| | | | | | C. | C. | | |
| | | | | | annuum, | annuum, | | |
| | | | | | A | A | | |
| | | | | | thaliana | thaliana, | | |
| | | | | | | S | | |
| | | | | | | lycopersicum | | |

| | Region II | | | | | | |
|---|---|---|---|---|---|---|---|
| Mutation | G99R | D101G | S161K | Q166K | G213D | G213S | G213L |
| Origine | P. | S | P. | H. | H. | H. | C |
| | sativum, | lycopersicum | sativum, | vulgare | vulgare | vulgare, | Melo |
| | C. | | A | | | S | |
| | annuum, | | thaliana | | | lycopersicum | |
| | A | | | | | | |
| | thaliana | | | | | | |

TABLE 3
Results of Y2H experiments on interaction between (i)
MDMV or SCMV and (ii) wild-type and eIF4E1 variants

| | MDMV | SCMV |
|---|---|---|
| eIF4E1 | VPg | VPg |
| ZmeIF4E1, WT | + | + |
| T7S | + | + |
| A19V | + | + |
| L33I | + | + |
| N55D | + | + |
| N55K | + | + |
| P56S | + | + |
| P56T | + | − |
| A63D | + | + |
| A64D | + | + |
| S67A | + | + |
| V96D | + | − |
| G97R | + | − |
| D99G | − | − |
| S159K | + | − |
| Q164K | + | + |
| G211D | + | + |
| G211L | + | + |
| G211S | + | + |
| T7S A19V | + | + |
| T7S A19V L33I | + | − |
| N55D P56S | + | + |
| N55K P56T | + | − |
| N55D P56T | + | − |
| N55K P56S | + | − |
| V96D G97R | − | − |
| A63D A64D | − | − |
| S67A S159K | + | − |
| D99G_Q164K | − | − |
| S159K_Q164K | + | − |
| N55K P56T G211S | + | − |
| N55K P56T S159K | + | − |
| A63D A64D G97R | − | − |
| A63D A64D D99G | − | − |
| A63D A64D S159K | − | − |
| S67A V96D G97R | + | − |
| V96D G97R D99G | − | − |
| V96D G97R S159K | − | − |
| A63D A64D G97R S159K | − | − |
| A63D A64D D99G S159K | − | − |
| A63D A64D S67A S159K | − | − |
| A63D A64D V96D G97R S159K | − | − |
| A63D A64D V96D G97R | − | − |
| N55K P56T A63D A64D | − | − |
| N55D P56S A63D A64D | − | − |
| N55D P56S A63D A64D G97R S159K | − | − |
| N55D P56S A63D A64D V96D G97R S159K | − | − |
| N55D P56S A63D A64D V96D G97R S159K G211S | − | − |
| N55K P56T A63D A64D V96D G97R S159K G211D | − | − |

“+”: interaction between VPg and eIF4E; “−” no interaction detected.

TABLE 4
Results of Y2H experiments on interaction between (i)
MDMV or SCMV and (ii) wild-type and elF4E2 variants

| eIF4E2 | MDMV VPg | SCMV VPg |
|---|---|---|
| ZmeIF4E2, WT | + | + |
| N57D | + | + |
| N57K | + | + |
| P58S | + | + |
| P58T | + | + |
| A65D | + | + |
| A66D | − | + |
| S69A | + | + |
| G98D | + | + |
| G99R | + | − |
| D101G | − | − |
| S161K | + | − |
| Q166K | + | + |
| G213D | + | + |
| G213S | + | + |
| G213L | + | + |
| N57D P58S | + | + |
| N57K P58T | + | + |
| N57D P58T | + | + |
| N57K P58S | + | + |
| A65D A66D | − | − |
| G98D G99R | + | − |
| S69A S161K | + | − |
| N57K P58T S161K | + | − |
| N57K P58T G213S | + | + |
| A65D A66D G99R | − | − |
| A65D A66D S161K | − | − |
| A65D A66D D101G | − | − |
| A65D A66D D101G S161K | − | − |
| G98D G99R D101G | − | − |
| G98D G99R S161K | + | − |
| S69A G98D G99R | + | − |
| A65D A66D G99R S161K | − | − |
| A65D A66D S69A S161K | − | − |
| A65D A66D G98D G99R | − | − |
| A65D A66D G98D G99R S161K | − | − |
| N57K P58T A65D A66D | − | − |
| N57D P58S A65D A66D | − | − |
| N57D P58S G98D G99R S161K | + | − |
| N57K P58T G98D G99R S161K G213D | + | − |
| D101G Q166K | − | − |
| D101G S161K Q166K G213L | − | − |
| S161K Q166K | + | − |

“+”: interaction between VPg and eIF4E; “−” no interaction detected.

With these results, a set of mutations coming from several species as source has been identify on eIF(iso)4E genes that disrupt interaction for both viruses, SCMV and MDMV. The list of validated mutations is:

| Name | Type |
|---|---|
| Zm00001d014065_{—} | A60D/A61D/N94R/W108L/S157K/R161E |
| eIFiso4E1_Variants | (sequence SEQ ID NO: 7) |
| Zm00001d032775_{—} | A54D/A55D/T88R/W102L/S151K/R155E |
| eIFiso4E2_Variants | (sequence SEQ ID NO: 8) |
| Zm00001d041682_{—} | A63D/A64D/V96D/G97R/D99G/G211L |
| eIF4E1_Variants | (sequence SEQ ID NO: 5) |
| Zm00001d041973_{—} | A65D/A66D/G98D/G99R/D101G/G213L |
| eIF4E2_Variants | (sequence SEQ ID NO: 6) |

### Example 4: Transgenic Expression of eIF(Iso)4E Variants in Maize J1A Genotype

Validation of maize variants of interest was done by transgenesis by expression ZmeIF4E variants in a susceptible background. The scientific basis relies on the failure of viruses to infect plants with an expression of eIF4E variants which results from both effect of the abundance of the eIF4E resistant allele, which the virus cannot recruit, and the inability to virus to access the product of the native susceptible allele due to competition with the resistant version. Expression of eIF4E1 variants has been chosen to have an over representation of the resistant alleles.

A set of 7 different constructs has been tested for eIF4E1:

| | |
|---|---|
| Zm00001d041682_eIF4E1_Native | (optimized) |
| Zm00001d041682_eIF4E1_Var1 | A63D/A64D |
| Zm00001d041682_eIF4E1_Var2 | D99G |
| Zm00001d041682_eIF4E1_Var3 | A63D/A64D/D99G |
| Zm00001d041682_eIF4E1_Var4 | V96D/G97R |
| Zm00001d041682_eIF4E1_Var5 | A63D/A64D/V96D/G97R |
| Zm00001d041682_eIF4E1_Var6 | A63D/A64D/V96D/G97R/D99G/ |
| | G211L |
| Zm00001d041682_eIF4E1_Var7 | G211L |

For each construct, 3 independent events were selected, and for each of them, 5 plants were infected by MDMV. Plants were grown on cultivation tables, growing conditions were 24° C. Day/19° C. Night with light cycle of 16h day/8h night. 13 days after sowing, artificial infection of the plants was performed by mechanic infection of the 2nd or 3rd leaf using fresh MDMV infected leaves ground in a phosphate buffer containing an abrasive. Scoring was done 21 days after infection and scoring was done from 0 (resistant) to 5 (susceptible).

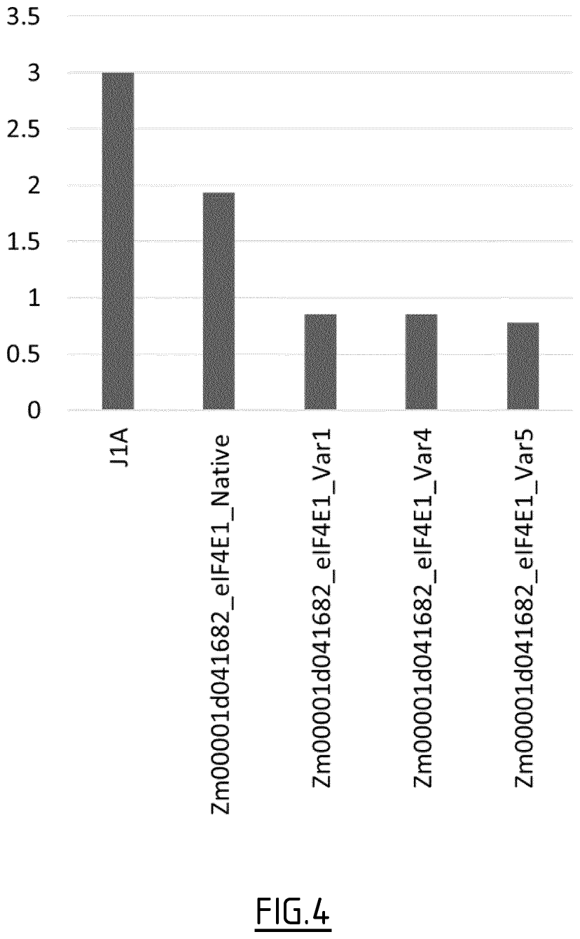

Expression of variants harboring mutations of interest identified by Y2H showed a clear reduction of infection rate by MDMV compared to J1A and with plants overexpressing the native allele (see for example FIG. 4). These results show the interest of these variants for virus resistance in maize.

### Example 5: Production of eIF4E Resistant Variants for Resistance to SCMV & MDMV (Potyviridae) and MCMV (Tombusviridae) in Maize by the Prime Editing Method

A susceptible plant containing eif4e gene is changed by genome editing into a resistant one. The modification of the amino acids in the positions listed in following table, was achieved by the prime editing methodology (Anzalone et al., 2019) using epegRNAs (enhanced prime editing guide RNAs) (Nelson et al., 2021). These epegRNAs had a 3′ tevoprecQ1 pseudo-knot sequence designed to stabilize the pegRNA (Nelson et al., 2021). The spacer, Primer binding site (PBS) and Reverse Transcriptase Template region (RTT) of the epegRNAs (Table 5) were designed with the aid of the PlantPegDesigner tool. Combinations of epegRNAs individually expressed from a maize U6 promoter (SEQ ID NO: 24) were cloned together with a ZmUbiquitin promoter-PE2 prime editor (SEQ ID NO: 25) into a plant binary vector containing a rice Actin promoter-: BAR selectable marker cassette. Binary vectors were transformed into an agrobacterium strain and maize transformation of the susceptible J1A variety performed essentially as described by Ishida et al., (2007). Edited plants with the desired nucleotides changes alone or in combination were identified by Next Generation sequencing of PCR amplicons covering the Prime Editing targeted sites.

| Name | Type |
|---|---|
| Zm00001d041682_{—} | A63D/A64D/V96D/G97R/D99G/G211L |
| eIF4E1_Variants | (sequence SEQ ID NO: 5) |
| Zm00001d041973_{—} | A65D/A66D/G98D/G99D/D101G/G213L |
| eIF4E2_Variants | (sequence SEQ ID NO: 6) |

TABLE 5
epegRNA designs

| | | | SEQ ID | |
|---|---|---|---|---|
| Exon | Change | Gene | NO: | epegRNA sequence (no promoter or terminator) |
| exon | A63D/ | eIF4E1 | 26 | GCGGATGGAGCTCCCCCAGGGTTTCAGAGCTATGCTGGAAAC |
| 1 | A64D | | | AGCATAGCAAGTTGAAATAAGGCTAGTCCGTTATCAACTTGAA |
| | | | | AAAGTGGCACCGAGTCGGTGCAGTCCAAGCAGGATGATTGGG |
| | | | | GGAGCTCTCTCTCCGCGGTTCTATCTAGTTACGCGTTAAACCAA |
| | | | | CTAGAA |
| exon | A63D/ | eIF4E1 | 27 | GGGGCGGATGGAGCTCCCCCGTTTCAGAGCTATGCTGGAAAC |
| 1 | A64D | | | AGCATAGCAAGTTGAAATAAGGCTAGTCCGTTATCAACTTGAA |
| | | | | AAAGTGGCACCGAGTCGGTGCAGTCCAAGCAGGATGATTGGG |
| | | | | GGAGCTCCATCTCTCTCCGCGGTTCTATCTAGTTACGCGTTAAA |
| | | | | CCAACTAGAA |
| exon | A63D/ | eIF4E1 | 28 | GCCCGCAGAGCAAGTCCAAGCGTTTCAGAGCTATGCTGGAAA |
| 1 | A64D | | | CAGCATAGCAAGTTGAAATAAGGCTAGTCCGTTATCAACTTGA |
| | | | | AAAAGTGGCACCGAGTCGGTGCGGAGCTCCCCCAATCATCCT |
| | | | | GCTTGGACTTGCTTCTCTCTCCGCGGTTCTATCTAGTTACGCGT |
| | | | | TAAACCAACTAGAA |
| exon | A65D/ | eIF4E2 | 29 | GTCGGATGGAGCTCCCCCAGGGTTTCAGAGCTATGCTGGAAA |
| 1 | A66D | | | CAGCATAGCAAGTTGAAATAAGGCTAGTCCGTTATCAACTTGA |
| | | | | AAAAGTGGCACCGAGTCGGTGCAAACAAGATGGATGATTGGG |
| | | | | GGAGCTCTCTCTCCGCGGTTCTATCTAGTTACGCGTTAAACCAA |
| | | | | CTAGAA |
| exon | A65D/ | eIF4E2 | 30 | GGGTCGGATGGAGCTCCCCCGTTTCAGAGCTATGCTGGAAAC |
| 1 | 6A6D | | | AGCATAGCAAGTTGAAATAAGGCTAGTCCGTTATCAACTTGAA |
| | | | | AAAGTGGCACCGAGTCGGTGCAAACAAGATGGATGATTGGG |
| | | | | GGAGCTCCATCTCTCTCCGCGGTTCTATCTAGTTACGCGTTAAA |
| | | | | CCAACTAGAA |
| exon | A65D/ | eIF4E2 | 31 | GCCCGCACGGCAAAAACAAGAGTTTCAGAGCTATGCTGGAAA |
| 1 | A66D | | | CAGCATAGCAAGTTGAAATAAGGCTAGTCCGTTATCAACTTGA |
| | | | | AAAAGTGGCACCGAGTCGGTGCGGAGCTCCCCCAATCATCCAT |
| | | | | CTTGTTTTTGCCGTCTCTCTCCGCGGTTCTATCTAGTTACGCGTT |
| | | | | AAACCAACTAGAA |
| exon | D99G | eIF4E1 | 32 | GCGATCTTGTTCTTGAAGCAAGTTTCAGAGCTATGCTGGAAAC |
| 2 | | | | AGCATAGCAAGTTGAAATAAGGCTAGTCCGTTATCAACTTGAA |
| | | | | AAAGTGGCACCGAGTCGGTGCGATCGTGGGAGCCGGATTCCA |
| | | | | TTGCTTCAAGAACATCTCTCTCCGCGGTTCTATCTAGTTACGCG |
| | | | | TTAAACCAACTAGAA |
| exon | D99G | eIF4E1 | 33 | GCACCCTAGCAAATTGATCGTGTTTCAGAGCTATGCTGGAAAC |
| 2 | | | | AGCATAGCAAGTTGAAATAAGGCTAGTCCGTTATCAACTTGAA |
| | | | | AAAGTGGCACCGAGTCGGTGCAAGCAATGGAATCCGGCTCCC |
| | | | | ACGATCAATTTGCTATCTCTCTCCGCGGTTCTATCTAGTTACGC |
| | | | | GTTAAACCAACTAGAA |
| exon | D99G | eIF4E1 | 34 | GTCACCCTAGCAAATTGATCGGTTTCAGAGCTATGCTGGAAAC |
| 2 | | | | AGCATAGCAAGTTGAAATAAGGCTAGTCCGTTATCAACTTGAA |
| | | | | AAAGTGGCACCGAGTCGGTGCAAGCAATGGAATCCGGCTCCC |
| | | | | ACGATCAATTTGCTAGTCTCTCTCCGCGGTTCTATCTAGTTACG |
| | | | | CGTTAAACCAACTAGAA |
| exon | D101G | eIF4E2 | 35 | GCAATCTTGTTCTTGAAGCAAGTTTCAGAGCTATGCTGGAAAC |
| 2 | | | | AGCATAGCAAGTTGAAATAAGGCTAGTCCGTTATCAACTTGAA |
| | | | | AAAGTGGCACCGAGTCGGTGCGATCGGCGGAGCCGGATTCCA |
| | | | | TTGCTTCAAGAACATCTCTCTCCGCGGTTCTATCTAGTTACGCG |
| | | | | TTAAACCAACTAGAA |
| exon | D101G | eIF4E2 | 36 | GCACCCTAGCAAGTTGATCGGGTTTCAGAGCTATGCTGGAAAC |
| 2 | | | | AGCATAGCAAGTTGAAATAAGGCTAGTCCGTTATCAACTTGAA |
| | | | | AAAGTGGCACCGAGTCGGTGCAAGCAATGGAATCCGGCTCCG |
| | | | | CCGATCAACTTGCTTCTCTCTCCGCGGTTCTATCTAGTTACGCG |
| | | | | TTAAACCAACTAGAA |
| exon | D101G | eIF4E2 | 37 | GCATCACCCTAGCAAGTTGATGTTTCAGAGCTATGCTGGAAAC |
| 2 | | | | AGCATAGCAAGTTGAAATAAGGCTAGTCCGTTATCAACTTGAA |
| | | | | AAAGTGGCACCGAGTCGGTGCAAGCAATGGAATCCGGCTCCG |
| | | | | CCGATCAACTTGCTAGGTCTCTCTCCGCGGTTCTATCTAGTTAC |
| | | | | GCGTTAAACCAACTAGAA |
| exon | V96D/ | eIF4E1 | 38 | GCACCCTAGCAAATTGATCGTGTTTCAGAGCTATGCTGGAAAC |
| 2 | G97R/D99G | | | AGCATAGCAAGTTGAAATAAGGCTAGTCCGTTATCAACTTGAA |
| | | | | AAAGTGGCACCGAGTCGGTGCAAGCAATGGAAACCGGCCCTA |
| | | | | TCGATCAATTTGCTATCTCTCTCCGCGGTTCTATCTAGTTACGC |
| | | | | GTTAAACCAACTAGAA |
| exon | V96D/ | eIF4E1 | 39 | GTCACCCTAGCAAATTGATCGGTTTCAGAGCTATGCTGGAAAC |
| 2 | G97R/D99G | | | AGCATAGCAAGTTGAAATAAGGCTAGTCCGTTATCAACTTGAA |
| | | | | AAAGTGGCACCGAGTCGGTGCAAGCAATGGAAACCGGCCCTA |
| | | | | TCGATCAATTTGCTAGTCTCTCTCCGCGGTTCTATCTAGTTACG |
| | | | | CGTTAAACCAACTAGAA |
| exon | V96D/ | eIF4E1 | 40 | GCGATCTTGTTCTTGAAGCAAGTTTCAGAGCTATGCTGGAAAC |
| 2 | 7G9R/D99G | | | AGCATAGCAAGTTGAAATAAGGCTAGTCCGTTATCAACTTGAA |
| | | | | AAAGTGGCACCGAGTCGGTGCAAATTGATCGATAGGGCCGGT |
| | | | | TTCCATTGCTTCAAGAACATCTCTCTCCGCGGTTCTATCTAGTT |
| | | | | ACGCGTTAAACCAACTAGAA |
| exon | G98D/ | eIF4E2 | 41 | GCACCCTAGCAAGTTGATCGGGTTTCAGAGCTATGCTGGAAAC |
| 2 | G99R/ | | | AGCATAGCAAGTTGAAATAAGGCTAGTCCGTTATCAACTTGAA |
| | D101G | | | AAAGTGGCACCGAGTCGGTGCAGCAATGGAAGCCGGCCCTGT |
| | | | | CGATCAACTTGCTTCTCTCTCCGCGGTTCTATCTAGTTACGCGT |
| | | | | TAAACCAACTAGAA |
| exon | G98D/ | eIF4E2 | 42 | GCATCACCCTAGCAAGTTGATGTTTCAGAGCTATGCTGGAAAC |
| 2 | G99R/ | | | AGCATAGCAAGTTGAAATAAGGCTAGTCCGTTATCAACTTGAA |
| | D101G | | | AAAGTGGCACCGAGTCGGTGCAGCAATGGAAGCCGGCCCTGT |
| | | | | CGATCAACTTGCTAGGTCTCTCTCCGCGGTTCTATCTAGTTACG |
| | | | | CGTTAAACCAACTAGAA |
| exon | G98D/ | eIF4E2 | 43 | GCAATCTTGTTCTTGAAGCAAGTTTCAGAGCTATGCTGGAAAC |
| 2 | G99R/ | | | AGCATAGCAAGTTGAAATAAGGCTAGTCCGTTATCAACTTGAA |
| | D101G | | | AAAGTGGCACCGAGTCGGTGCAAGTTGATCGACAGGGCCGGC |
| | | | | TTCCATTGCTTCAAGAACATCTCTCTCCGCGGTTCTATCTAGTT |
| | | | | ACGCGTTAAACCAACTAGAA |
| exon | G211L/ | eIF4E1/ | 44 | GGATGCAAAGAAGATGGACAAGTTTCAGAGCTATGCTGGAAA |
| 5 | G213L | eIF4E2 | | CAGCATAGCAAGTTGAAATAAGGCTAGTCCGTTATCAACTTGA |
| | | | | AAAAGTGGCACCGAGTCGGTGCACGGTTCTTCAGCAGCTTGTC |
| | | | | CATCTTCTTTCTCTCTCCGCGGTTCTATCTAGTTACGCGTTAAAC |
| | | | | CAACTAGAA |
| exon | G211L/ | eIF4E1/ | 45 | GGCAGGACGATGCAAAGAAGAGTTTCAGAGCTATGCTGGAAA |
| 5 | G213L | eIF4E2 | | CAGCATAGCAAGTTGAAATAAGGCTAGTCCGTTATCAACTTGA |
| | | | | AAAAGTGGCACCGAGTCGGTGCACGGTTCTTCAGCAGCTTGTC |
| | | | | CATCTTCTTTGCATCGTCTCTCTCCGCGGTTCTATCTAGTTACGC |
| | | | | GTTAAACCAACTAGAA |
| exon | G211L | eIF4E1 | 46 | GAAACCGTGTAACGGTTCTTCGTTTAAGAGCTAGAAATAGCAA |
| 5 | | | | GTTTAAATAAGGCTAGTCCGTTATCAACTTGAAAAAGTGGCAC |
| | | | | CGAGTCGGTGCAAGATGGACAAGCTGCTGAAGAACCGTTACT |
| | | | | CTCTCTCCGCGGTTCTATCTAGTTACGCGTTAAACCAACTAGAA |
| exon | G211L | eIF4E2 | 47 | GCGATGCAAAGAAGATGGACAGTTTCAGAGCTATGCTGGAAA |
| 5 | | | | CAGCATAGCAAGTTGAAATAAGGCTAGTCCGTTATCAACTTGA |
| | | | | AAAAGTGGCACCGAGTCGGTGCACGGTTCTTCAGCAGCTTGTC |
| | | | | CATCTTCTTTTCTCTCTCCGCGGTTCTATCTAGTTACGCGTTAAA |
| | | | | CCAACTAGAA |

### Example 6: Phenotyping of GE (Genetically Edited) Plants after Viral Infection by SCMV, MDMV and/or MCMV

GE (genetically edited) plants were phenotyped in greenhouse to evaluate resistance to 3 different viruses: SCMV & MDMV (Potyviridae) and MCMV (Tombusviridae) alone or by combining MCMV and a potyviruse to mimic Maize Lethal Necrosis Disease. Plants were grown on separate cultivation tables for each modality of viral infection (5 in total) to avoid unwanted virus cross-contamination. Growing condition were 24° C. Day/19° C. Night with light cycle of 16h day/8h night. 13 days after sowing, artificial infection of the plants was performed by mechanic infection of the 2nd or 3rd leaf using fresh infected leaves ground in a phosphate buffer containing an abrasive.

### Example 7: ELISA Tests to Monitor Presence/Absence of Virus

ELISA tests were done on leaf sampling done at 15 DPI to monitor and quantify viruses used for infection (SCMV, MDMV and MCMV). ELISA can measure the degree of virus accumulation using anti-SCMV/MDMV/MCMV antibodies in plants. In the GE/GM maize plants with a eIF4E protein variant, level of viruses has been compared to wild-type maize plants.

1. An eIF(iso)4E protein variant able to confer resistance to at least one maize virus, wherein said protein variant comprises at least one mutation by comparison to the corresponding native maize eIF(iso)4E.

2. The eIF(iso)4E protein variant according to claim 1, wherein the eIF(iso)4E protein variant is an eIF4E1 protein variant, an eIF4E2 protein variant, an eIFiso4E1 protein variant or an eIFiso4E2 protein variant.

3. The eIF(iso)4E protein variant according to claim 1, wherein said native maize eIF(iso)4E protein is protein eIF4E1 of sequence SEQ ID NO: 1, protein eIF4E2 of sequence SEQ ID NO: 2, protein eIFiso4E1 of sequence SEQ ID NO: 3 or protein eIFiso4E2 of sequence SEQ ID NO: 4.

4. The eIF(iso)4E protein variant according to claim 1, wherein said maize virus is a potyvirus or a machlomovirus.

5. The eIF(iso)4E protein variant according to claim 1, wherein said at least one mutation disrupts the interaction between said protein variant and at least one virus selected from the group consisting of MCMV (Maize Chlorotic Mottle Virus), SCMV (Sugarcane Mosaic Virus), MDMV Maize (Dwarf Mosaic Virus) and WSMV (Wheat Streak Mosaic Virus).

6. The eIF(iso)4E protein variant according to claim 1, wherein said at least one mutation is selected from:
214LT: 0000:583971:1:ALEXANDRIA
the group consisting of V96D, G97R, T7S, A19V, L33I, N55D/K, P56S/T, A63D, A64D, S67A, D99G, S159K, Q164K and G211L/D/S, by reference to native maize protein of sequence SEQ ID NO: 1,
the group consisting of D101G, A65D, A66D, N57D/K, P58S/T, S69A, G98D, G99R, S161K, Q166K and G213L/D/S, by reference to native maize protein of sequence SEQ ID NO: 2,
the group consisting of A60D, A61D, N94R, W108L, S157K and R161E/L, by reference to native maize protein of sequence SEQ ID NO: 3, or
the group consisting of A54D, A55D, T88R, W102L, S151K and R155E/L, by reference to native maize protein of sequence SEQ ID NO: 4.

7. The eIF(iso)4E protein variant according to claim 1, wherein said protein variant comprises at least two mutations by comparison to the corresponding native maize eIF(iso)4E protein.

8. The eIF(iso)4E protein variant according to claim 1, wherein said protein variant comprises:
mutations (i) A63D and A64D, (ii) V96D and G97R, (iii) D99G and/or (iv) G211L/D/S, by reference to native maize protein of sequence SEQ ID NO: 1,
mutations (i) A65D and A66D, (ii) G98D and G99R, (iii) D101G and/or (iv) G213L/D/S, by reference to native maize protein of sequence SEQ ID NO: 2,
mutations (i) A60D and A61D, (ii) N94R, (iii) W108L and R161E/L, and/or (iv) S157K, by reference to native maize protein of sequence SEQ ID NO: 3, or
mutations (i) A54D, A55D, (ii) T88R, (iii) W102L and R155E/L and/or (iv) S151K, by reference to native maize protein of sequence SEQ ID NO: 4.

9. A nucleic acid encoding at least one eIF(iso)4E protein variant according to claim 1.

10. A vector comprising at least one nucleic acid encoding at least one eIF(iso)4E protein variant according to claim 1.

11. A genetically modified or edited maize plant resistant to at least one maize virus or a genetically modified or edited maize seed resistant to at least one maize virus, wherein said plant or seed comprises at least one eIF(iso)4E protein variant according to claim 1 and wherein said plant is not obtained by means of an essentially biological process.

12. A progeny of the genetically modified or edited plant according to claim 11.

13. A method for obtaining a genetically modified or edited maize plant resistant to at least one maize virus, wherein said method comprises:
transforming a maize cell or maize tissue with at least one vector according to claim 10, to obtain a transformed maize cell or a transformed maize tissue, and
regenerating a maize plant from the transformed maize cell or transformed maize tissue.

14. A method for obtaining an edited modified maize plant resistant to at least one maize virus, wherein said method comprises introducing at least one mutation in an endogenous maize gene encoding an eIF(iso)4E protein, to obtain a mutated gene encoding an eIF(iso)4E protein variant according to claim 1.

15. A method for preventing at least one viral disease in maize, wherein said method comprises expressing at least one eIF(iso)4E protein variant according to claim 1 in said maize.

16. A method for identifying a maize plant resistant to at least one maize virus, wherein said method comprises detecting in the cells of a maize plant the expression of an eIF(iso)4E protein variant claim 1 and/or the presence of a nucleic acid encoding an eIF(iso)4E protein variant according to claim 1.
